(19) United States
(12) Patent Application Publication
(10) Pub. No.: US 2025/0230195 A1
(43) Pub. Date: Jul. 17, 2025

(54) PREFUSION STABILIZED EBV GB MUTATIONS AND USES THEREOF

(71) Applicant: Seattle Children’s Hospital dba Seattle Children’s Research Institute, Seattle, WA (US)

(72) Inventors: Jason Price, Seattle, WA (US); Bianka Haro, Seattle, WA (US); Raymond Ruff, Seattle, WA (US); Jim Olson, Seattle, WA (US)

(73) Assignee: Seattle Children’s Hospital dba Seattle Children’s Research Institute, Seattle, WA (US)

(21) Appl. No.: 18/876,289

(22) PCT Filed: Jun. 29, 2023
(86) PCT No.: PCT/US2023/069411

§ 371 Date: Dec. 18, 2024

Related U.S. Application Data
(60) Provisional application No. 63/356,756, filed on Jun. 29, 2022.

(51) Int. Cl. C07K 14/005; A61K 39/245

(52) U.S. Cl. CPC C07K 14/005; A61K 39/245; C07K 2319/02; C07K 2319/21; C12N 2710/16222; C12N 2710/16234

(57) ABSTRACT

We have generated a 3D model of the glycoprotein B (gB) of Epstein-Barr virus (EBV) to design candidate stabilizing mutations that increase the stability of the prefusion state essential for an effective EBV gB based vaccine. Provided herein are engineered polypeptides derived from the EBV gB, which include an altered EBV gB ectodomain that has modifications relative to the native EBV gB ectodomain that stabilize a prefusion conformation of the polypeptides. In various aspects, the modifications are amino acid substitutions to generate pairs of cysteine amino acid residues, preferably positioned to connect different domains of the poly peptide or different copies of the polypeptide in a trimeric or multimeric conformation via formation of disulfide bonds during protein expression. In additional aspects, the modifications and/or the engineered polypeptides do not contain pairs of cysteine amino acid residues that may form disulfide bonds in a postfusion conformation.

### CROSS-REFERENCE TO RELATED APPLICATIONS

This application includes a claim of priority under 35 U.S.C. § 119(e) to U.S. provisional patent application No. 63/356,756, filed Jun. 29, 2022, the entirety of which is hereby incorporated by reference.

### REFERENCE TO SEQUENCE LISTING

This application contains a Sequence Listing submitted as an electronic file named “067505_000009WOPT_SequenceListing.xml”, having a size in bytes of 69,405 bytes, and created on Jun. 29, 2023 (WIPO production date noted as 2023 Jun. 29). The information contained in this electronic file is hereby incorporated by reference in its entirety.

### FIELD OF INVENTION

This invention relates to engineered glycoprotein B variants of Epstein-Barr virus, especially cysteine engineered variants for disulfide formation and stabilization in a prefusion conformation, and related immunogenic compositions.

### BACKGROUND

Epstein-Barr virus (EBV) is a member of the herpes virus family and is associated with development of malignancies of lymphoid tissue, such as causing mononucleosis and contributing to certain cancer and autoimmune diseases. Without wishing to be bound by a particular theory, EBV, a γ-herpesvirus, enters epithelial cells and cultured B cells by direct fusion with the cell membrane, whereas endocytosis appears required for efficient infection of primary human lymphocytes. Regardless of the entry pathway, fusion of the viral and cell membranes has to occur to allow the release of virion capsids into the cytoplasm prior to the genome being replicated in the nucleus. The fusion apparatus of EBV is comprised of envelope glycoprotein B (gB) and a heterodimeric complex made of glycoproteins H and L (gH/gL). In addition, EBV may also express glycoproteins, such as gp42, that determine viral tropism in the family of herpesviruses and mediate receptor-dependent activation of membrane fusion.

The EBV gB ectodomain is an elongated rod-like molecule, composed of 5 domains that contain both β-sheet and a-helical secondary structures (FIG. 1A). A trimeric gB ectodomain may form when three gB subunits wrap around each other by interacting through multiple contact surfaces to form a spike-like trimer (FIG. 1B). Backovic et al. described in Proc Natl Acad Sci USA 2009 Feb. 24; 106(8): 2880-2885 that Domain I (residues 89-294 in Swiss-Prot entry number PO3188) contains the gB fusion loops (FLs), and its core region has a fold that resembles that of a plekstrin-homology (PH) domain; Domain II is composed of residues 77-88 and 295-390, and has a PH domain fold; Domain III is the 42-residue long αC helix, which wraps around the helices from other 2 subunits in a left-handed twist, and is composed of residues 52-68, 455-527, and 617-624; Domain IV is made of residues 528-616 and a short N-terminal region consisting of residues 42-51; and Domain V is composed of residues 625-679, which reaches across and inserts into the cavity formed by other 2 subunits, tying together the gB trimer. The domain structures found in gB, VSV G, and in the baculovirus fusion protein gp64, led to their classification as a class III type of viral fusion protein.

Although EBV is one of the most common human viruses, there is no specific treatment for EBV. To date, it has been difficult to develop a vaccine against EBV for a variety of reasons including the inability to select appropriate antigen targets for vaccine development, lack of an appropriate vaccine platform, and lack of an appropriate evaluation system for EBV vaccine assessment.

In view of the limitations of the present art for developing an approved EBV vaccine, a need remains for a prophylactic EBV vaccine or an immunogenic composition that would provide protection against infectious diseases and EBV-associate malignancies.

Therefore, it is an objective of the present invention to provide polypeptides and/or polynucleic acids, preferably as an isolated immunogen.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Various embodiments provide polypeptides. In various aspects, the polypeptides are engineered variants of the glycoprotein B (gB) of Epstein-Barr virus (EBV). In various aspects, the polypeptides are variants of the EBV gB with amino acid residue substitutions relative to the wildtype EBV gB, such that one or more disulfide bonds are formed in a prefusion conformation upon protein expression. In various aspects, the amino acid residue substitutions do not lead to formation of disulfide bonds in a postfusion conformation. In further aspects, the polypeptides do not have disulfide bonds in a postfusion conformation.

Various embodiments provide a polypeptide, which includes an altered gB ectodomain sequence that has modifications relative to native gB ectodomain sequence of the EBV, said native gB ectodomain referring to the amino acid sequence set forth as residues 23-683 of SEQ ID NO:1. In various aspects, the modifications include substituting one or more pairs of amino acid residues with one or more pairs of engineered sulfhydryl-containing amino acid residues. In various aspects, the modifications include substituting one or more pairs of amino acid residues with one or more pairs of cysteine amino acid residues.

In some embodiments, the modifications include amino acid substitutions of at least two of S54C, S55C, H56C, G172C, A175C, V178C, G227C, G322C, S325C, D478C, A480C, A515C, V529C, D564C, L580C, Y644C, L696C, and S727C compared to SEQ ID NO: 1, wherein the amino acid numbering is based on amino acid positions in SEQ ID NO:1. In further embodiments, the modifications include one or two or three or more pairs of amino acid substitutions including S54C and A515C, S55C and G227C, H56C and G227C, A175C and V529C, G322C and A480C, S325C and A480C, G322C and D478C, L696C and S727C, A175C and V178C, G172C and D564C, and L580C and Y644C, wherein the amino acid numbering is based on amino acid positions in SEQ ID NO:1. In various embodiments, native cysteine amino acid residues in close-by locations in a postfusion conformation are substituted with a non-Cysteine amino acid residues, such as Ala, Arg, As, Asp, Glu, Gln, Gly, His, Ile, Leu, Lys, Met, Phe, Pro, Ser, Thr, Trp, Tyr, and Val.

In some embodiments, the polypeptides relative to wild type EBV gB (or relative to wild type EBV gB ectodomain) further include amino acid substitutions in fusion loop. In further embodiments, the amino acid substitutions in fusion loop region include substituting WY¹¹²⁻¹¹³ and WLIW¹⁹³⁻¹⁹⁶ (SEQ ID NO:28) with HR and RVEA (SEQ ID NO: 29), wherein the amino acid numbering is based on amino acid positions in SEQ ID NO: 1.

In some embodiments, the polypeptides further include a signal sequence. In some embodiments, the polypeptides further include a trimerization sequence or multimerization sequence. In some embodiments, the polypeptides further include a purification tag, such as a polyhistidine sequence. For example, the polypeptides are in a configuration from the N-terminal to C-terminal direction: signal sequence-altered gB ectodomain sequence-trimerization domain-poly histidine sequence. Linker peptide sequences may be included, for example, in a configuration from the N-to-C terminal direction: signal sequence-altered gB ectodomain sequence-linker sequence 1-trimerization domain-linker sequence 2-poly histidine affinity tag.

Various embodiments provide proteins, wherein the protein is formed from three or more of the engineered polypeptides. In various aspects, a protein includes three copies of an engineered polypeptide. In further aspects, a protein includes a trimer or multimer formed from three or more copies of an engineered polypeptide, and further includes an additional polypeptide. In some aspects, the additional polypeptide is an EBV glycoprotein other than gB. For example, the additional polypeptide is EBV gHgL, gp42, gp350, gp220, complex gp350/220, or a combination thereof.

Various embodiments provide nucleic acid molecules encoding one or more of the engineered polypeptides. In some embodiments, a messenger ribonucleic acid (mRNA) molecule is provided, which contains an open reading frame encoding one or more engineered polypeptides. Vectors are also provided comprising the nucleic acid molecules. Isolated host cell comprising the vectors are included.

Various embodiments provide immunogenic compositions, including the engineered polypeptides, a protein formed from the engineered polypeptides, a nucleic acid molecule encoding the engineered polypeptides such as an mRNA molecule containing an open reading frame, or a combination of the above, further including a pharmaceutical acceptable excipient.

Methods of inducing an immunogenic response to an EBV protein are also provided. In some embodiments, a method to induce an immunogenic response includes administering an engineered polypeptide, a protein formed from or including the engineered polypeptide, and/or a nucleic acid molecule encoding the engineered polypeptide to a subject in need thereof. In some embodiments, the subject is a pediatric subject.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A depicts 3D models generated by Colabfold of full length EBV gB ectodomain monomer including transmembrane domain in the prefusion and postfusion state/conformation. The individual domains are labeled by color: the 5 domains of EBV gB are indicated with roman numbers I to V, and are defined and labeled after nomenclature established for the HSV-1 gB ectodomain consistent with Proc Natl Acad Sci USA 2009 Feb. 24; 106(8): 2880-2885. Alignment of the domains by the central helix (DIII) reveals the significant structural rearrangement between the two conformations.

FIG. 1B depicts 3D model generated by Colabfold of full-length wildtype EBV gB ectodomain trimer including transmembrane domain.

FIG. 1C depicts a cartoon representation of Colabfold-generated 3D model of full-length EBV gB ectodomain trimer including transmembrane domain, (purple), which is fit into the prefusion electron microscopy (EM) map of HSV1 gB (grey mesh).

FIG. 2 depicts exemplary formed disulfide mutant pairings modeled with colabfold, and summarizes positions of engineered cysteine mutations, as well as the resulting interdomain/interchain disulfide bond crosslinked domains stabilizing the prefusion conformation. The chart lists selected cysteine mutant pairings predicted to stabilize prefusion-specific interdomain interactions. Mutations were selected by two ways: 1) manual inspection of the colabfold generated prefusion and postfusion 3D models in pymol. 2) using tool to identify candidate disulfide pairings in prefusion and postfusion models. Prefusion disulfide pairings that were identified/indicated present in the postfusion model, that were not interdomain interchain connecting, or that did not pass manual inspection, were eliminated.

FIG. 3 depicts exemplary formed disulfide mutant pairings in an engineered ectodomain construct (e.g., SEQ ID NOs:14-21) modeled by computational techniques, and summarizes the resulting interdomain/interchain disulfide bond crosslinked domains stabilizing the prefusion conformation.

FIGS. 4A-4B show results from purification with high-performance liquid chromatography (HPLC) for several engineered peptides.

FIGS. 5A-5B show results from purification with ultra-high performance liquid chromatography-size exclusion chromatography (UPLC-SEC) for several engineered peptides.

FIGS. 6A-6C show disulfide stabilized EBV gB variants from HPLC-SEC purified product when run in sodium dodecyl-sulfate polyacrylamide gel electrophoresis (SDS-PAGE) in non-reducing (NR) and reducing (R) conditions.

FIGS. 7A-7E show results of negative stain electron microscopy (EM) of purified product of disulfide stabilized EBV gB variants. 1% uranyl formate was used as the negative staining solution and Formvar/carbon grids were used.

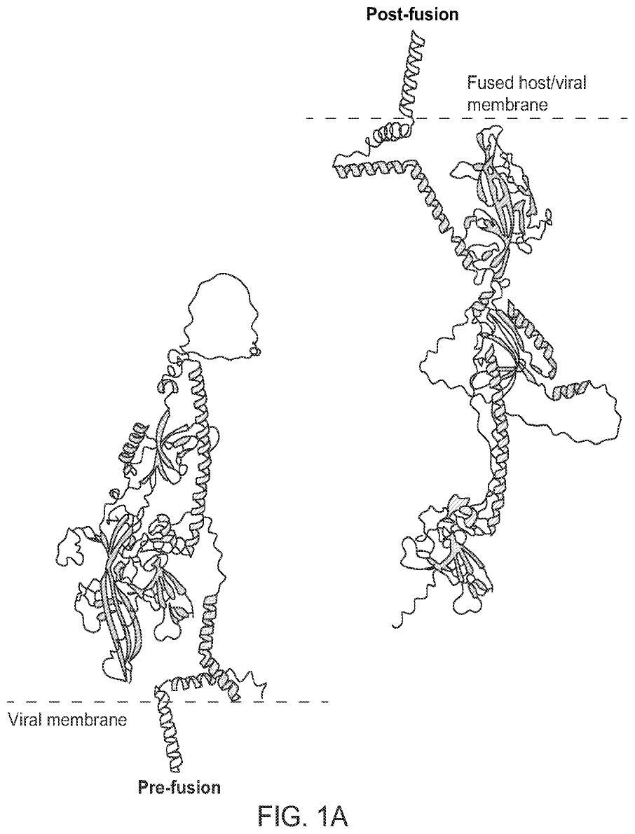

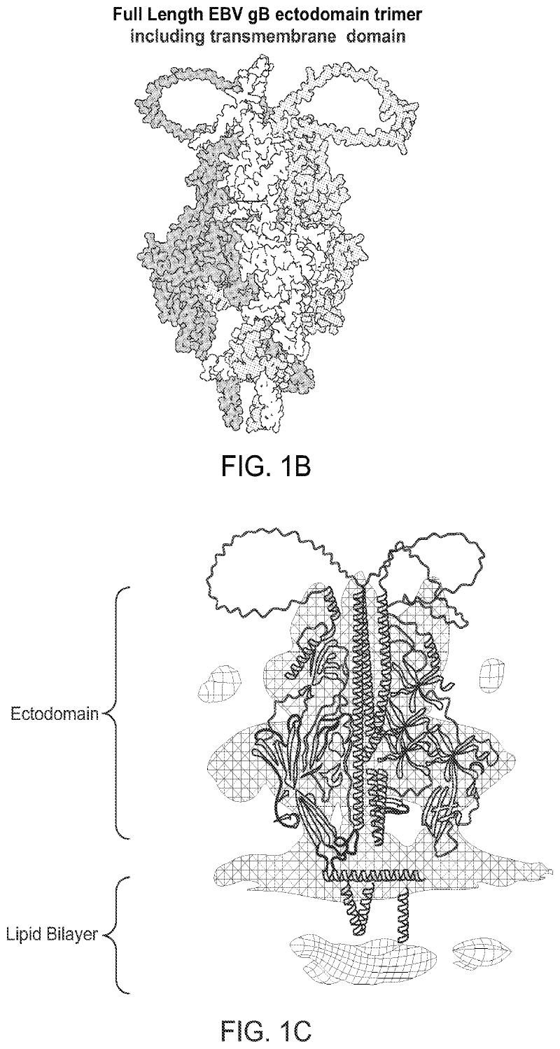

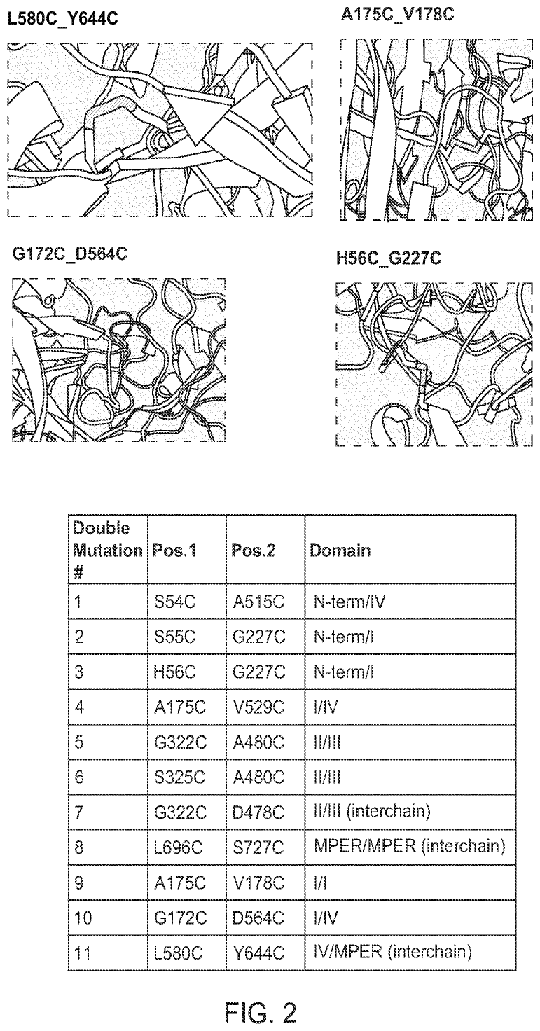

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Glycoprotein (gp)” generally refers to a protein that contains oligosaccharide chains (glycans) covalently attached to polypeptide side-chains. The carbohydrate is attached to the protein in a cotranslational or posttranslational modification, known as a process of glycosylation. In proteins that have segments extending extracellularly, the extracellular segments are often glycosylated. Glycoproteins are often important integral membrane proteins, where they play a role in cell-cell interactions. In some examples the polypeptides disclosed herein are a glycoprotein, such as an EBV gB protein variant stabilized in a prefusion conformation or an immunogenic fragment thereof.

“Glycosylation site” refers to an amino acid sequence on the surface of a polypeptide, such as a protein, which accommodates the attachment of a glycan. An N-linked glycosylation site is triplet sequence of NX(S/T) in which Nis asparagine, X is any residues except proline, and (S/T) is a serine or threonine residue. A glycan is a polysaccharide or oligosaccharide. Glycan may also be used to refer to the carbohydrate portion of a glycoconjugate, such as a glycoprotein, glycolipid, or a proteoglycan.

“Foldon” is a part of a protein that folds and unfolds as a unit. In various embodiments, foldon is the C-terminal domain of T4 fibritin, which is a trimerization domain.

“Ectodomain” is the domain of a membrane protein that extends into the extracellular space (the space outside a cell).

“Administration” is the introduction of a composition into a subject by a chosen route. Administration can be local or systemic. For example, if the chosen route is intravenous, the composition (such as a composition including a disclosed immunogen) is administered by introducing the composition into a vein of the subject. Exemplary routes of administration include but are not limited to intramuscular injection, intravenous injection, subcutaneous injection, intranasal administration, and oral administration.

Amino acid substitutions refer to the replacement of one amino acid with a different amino acid. Conservative amino acid substitutions providing functionally similar amino acids are well known in the art. The following six groups each contain amino acids that are conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

A conservative variant of a protein can be one with conservative substitutions, which results in at least 100%, 99%, 98%, 97%, 96%, 95%, 94%, 93%, 92%, 91%, or 90% of the function of the protein compared to the native protein. In other aspects, one of ordinary skill will recognize that individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

“Degenerate variant” refers to a polynucleotide encoding a polypeptide or an antibody include a sequence that is degenerate as a result of the genetic code. For example, a polynucleotide encoding a disclosed antigen or an antibody that specifically binds a disclosed antigen includes a sequence that is degenerate as a result of the genetic code. There are 20 natural amino acids, most of which are specified by more than one codon. Therefore, all degenerate nucleotide sequences are included as long as the amino acid sequence of the antigen or antibody that binds the antigen encoded by the nucleotide sequence is unchanged. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified within a protein encoding sequence, the codon can be altered to any of the corresponding codons described without altering the encoded protein. Such nucleic acid variations are “silent variations,” which are one species of conservative variations. Each nucleic acid sequence herein that encodes a polypeptide also describes every possible silent variation. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

“Immunogen” generally refers to a protein or a portion thereof, or a nucleic acid molecule encoding the protein, that is capable of inducing an immune response in a mammal, such as a mammal infected or at risk of infection with a pathogen. Administration of an immunogen can lead to protective immunity and/or proactive immunity against a pathogen of interest.

EBV is one of the most common viruses. Current treatment options do not include an approved vaccine. Disclosed herein include EBV glycoprotein B (gB) variants which have pairs of cysteine substitutions relative to native EBV gB, and the pairs of cysteine substitutions when linked through a disulfide bond can stabilize the EBV gB variants in a prefusion conformation. This prefusion conformation represents a new structure for development of EBV gB vaccines.

Various embodiments provide polypeptides derived from the glycoprotein B (gB) of an Epstein-Barr virus (EBV). In multiple instances, polypeptides derived from the gB of EBV are also referred to as engineered polypeptides. In various embodiments, the polypeptide derived from the EBV gB comprises an altered gB ectodomain sequence that has modifications relative to native gB ectodomain sequence of the EBV, wherein the native gB ectodomain comprising the amino acid sequence set forth as residues 23-683 of SEQ ID NO: 1, wherein the modifications comprise substituting one or more pairs of amino acid residues of the native gB ectodomain sequence with one or more pairs of sulfhydryl-containing amino acid residues. The amino acid sequence set forth as residues 23-683 of SEQ ID NO: 1 is a short ectodomain (EctoS) sequence, also individually listed as SEQ ID NO:27.

In various aspects, the modifications stabilize the polypeptide derived from the EBV gB in a prefusion conformation. In various aspects, the one or more pairs of sulfhydryl-containing amino acid residues each form a disulfide bond. In various aspects, the one or more pairs of sulfhydryl-containing amino acid residues are cysteine residues. In various aspects, the one or more pairs of sulfhydryl-containing amino acid residues, or pairs of cysteine residues, each form a disulfide bond in a prefusion conformation of the polypeptide. In various aspects, the one or more pairs of sulfhydryl-containing amino acid residues, or pairs of cysteine residues, each form a disulfide bond in a prefusion conformation of the polypeptide but not in a post-fusion conformation.

In various aspects, the one or more pairs of sulfhydryl-containing amino acid residues, or pairs of cysteine residues, form disulfide bridges connecting two different domains (in secondary structure of) within an EBV gB subunit (monomer), or between two different subunits (at a same or different domain) of a trimeric EBV gB. In various aspects, the one or more pairs of sulfhydryl-containing amino acid residues, or pairs of cysteine residues, do not form disulfide bridges within any one of the five domains in the secondary structure of a single EBV gB subunit. The five domains in the secondary structure of an EBV gB subunit, i.e., Domains I-V, as well as post-fusion and pre-fusion conformations, are described in Proc Natl Acad Sci USA 2009 Feb. 24; 106(8): 2880-2885 and in the Background; the sequence of EBV gB, i.e., Swiss-Prot entry number P03188, in Proc Natl Acad Sci USA 2009 Feb. 24; 106(8): 2880-2885 is identical to SEQ ID NO:1 herein. The five domains are located within the EctoS sequence (SEQ ID NO:27, or positions 23-683 of SEQ ID NO: 1). Generally, the substitutions with one or more pairs of the cysteine amino acids results in a polypeptide when produced recombinantly form disulfide bonds natively during secretion of recombinant protein from mammalian cells. Generally formed disulfide bonds are stable in aqueous buffer at physiological pH and temperature.

In various aspects, if one or more amino acid residues upon substitution with cysteine residues are close together in a postfusion conformation, either among the two of them or one of them with a wildtype cysteine residue, these amino acid residues are not substituted with cysteine residues. In various aspects, if native cysteine amino acid residues are close together in a postfusion conformation, at least one of the native amino acid residues is substituted with a non-sulfhydryl containing amino acid residue, such as Ala, Arg, As, Asp, Glu, Gln, Gly, His, Ile, Leu, Lys, Met, Phe, Pro, Ser, Thr, Trp, Tyr, and Val. See Table 1.

TABLE 1
Pairs that are predicted to form disulfide bridges (if substituted
with or retain both native cysteine amino acid residues) in
a postfusion conformation and thus not selected for prefusion
stabilization. All amino acid numbering is based on amino acid
positions in SEQ ID NO: 1. These pairs are not selected for
substitutions with cysteine amino acid residues or they require
substitutions with non-Cysteine amino acid residues.

| | |
|---|---|
| 49 ARG | 528 CYS |
| 51 CYS | 515 ALA |
| 51 CYS | 528 CYS |
| 68 CYS | 484 CYS |
| 82 LEU | 306 THR |
| 99 TYR | 127 VAL |
| 107 LEU | 119 ASN |
| 110 ASN | 191 PRO |
| 141 CYS | 206 CYS |
| 143 ASN | 158 ASP |
| 144 ALA | 163 ASN |
| 158 ASP | 204 VAL |
| 168 LEU | 181 TYR |
| 183 SER | 206 CYS |
| 211 MET | 225 THR |
| 215 SER | 222 PHE |
| 231 GLU | 245 HIS |
| 233 SER | 242 GLU |
| 236 TYR | 242 GLU |
| 247 ARG | 250 SER |
| 295 CYS | 342 CYS |
| 365 GLY | 384 PRO |
| 374 THR | 378 LEU |
| 418 ALA | 419 ARG |
| 419 ARG | 420 GLY |
| 420 GLY | 421 SER |
| 506 MET | 516 ALA |
| 535 THR | 560 SER |
| 539 ARG | 556 LEU |
| 540 LYS | 610 HIS |
| 547 SER | 550 MET |
| 551 CYS | 588 CYS |
| 589 GLN | 592 SER |

In various embodiments, relative to the native EBV gB ectodomain, the altered EBV gB ectodomain sequence does not have amino acid substitutions of any one of (1) R49C, while wild type residue C528 remains unchanged; (2) A515C, while wild type residue C51 remains unchanged; (3) L82C and T306C; (4) Y99C and V127C; (5) L107C and N119C; (6) N110C and P191C; (7) N143C and D158C; (8) A144C and N163C; (9) D158C and V204C; (10) L168C and Y181C; (11) S183C, while wild type residue C206 remains unchanged; (12) M211C and T225C; (13) S215C and F222C; (14) E231C and H245C; (15) S233C and E242C; (16) Y236C and E242C; (17) R247C and S250C; (18) G365C and P384C; (19) T374C and L378C; (20) A418C and R419C; (21) R419C and G420C; (22) G420C and S421C; (23) M506C and A516C; (24) T535C and S560C; (25) R539C and L556C; (26) K540C and H610C; (27) S547C and M550C; or (28) Q589C and S592C, wherein all amino acid numbering in (1)-(28) is based on amino acid positions in SEQ ID NO: 1.

In various embodiments, relative to the native EBV gB ectodomain, the altered EBV gB ectodomain sequence has modifications comprising substituting one or both native cysteine amino acid residues with another amino acid residues that does not contain a sulfhydryl group in each and all pairs, or any one, two, three, or four pairs, of (i) C51 and C528; (ii) C68 and C484; (iii) C141 and C206; (iv) C295 and C342; and (v) C551 and C588, wherein all amino acid number in (i)-(v) is based on amino acid positions in SEQ ID NO:1. In further embodiments, relative to the native EBV gB ectodomain, the altered EBV gB ectodomain sequence has modifications comprising substituting one or both native cysteine amino acid residues with another amino acid residues selected from the group of Ala, Arg, As, Asp, Glu, Gln, Gly, His, Ile, Leu, Lys, Met, Phe, Pro, Ser, Thr, Trp, Tyr, and Val in the pair of (i) C51 and C528; (ii) C68 and C484; (iii) C141 and C206; (iv) C295 and C342; and/or (v) C551 and C588, wherein all amino acid number in (i)-(v) is based on amino acid positions in SEQ ID NO:1.

To facilitate prefusion stabilization in various embodiments, at least two cysteine amino acid residues can be substituted at positions selected from S54, S55, H56, G172, A175, V178, G227, G322, S325, D478, A480, A515, V529, D564, L580, Y644, L696, and S727 that correspond to the native amino acid positions in the sequence of SEQ ID NO: 1. In some embodiments, the substitutions with the one or more pairs of engineered sulfhydryl-containing amino acid residues comprises amino acid substitutions of at least two of S54C, S55C, H56C, G172C, A175C, V178C, G227C, G322C, S325C, D478C, A480C, A515C, V529C, D564C, L580C, Y644C, L696C, and S727C compared to SEQ ID NO:1, wherein the amino acid numbering is based on amino acid positions in SEQ ID NO: 1.

In some embodiments, at least two cysteine amino acid residues can be substituted at position S54 and at position A515 based on amino acid positions in SEQ ID NO: 1. In some embodiments, the substitutions with the one or more pairs of engineered sulfhydryl-containing amino acid residues comprise amino acid substitutions of S54C and A515C compared to SEQ ID NO: 1 based on amino acid positions in SEQ ID NO:1. In some embodiments, the polypeptide having the modifications of S54C and A515C amino acid substitutions based on amino acid positions in SEQ ID NO:1 has or consists of an amino acid sequence of SEQ ID NO: 2. In some embodiments, the polypeptide having the modifications of S54C and A515C amino acid substitutions based on amino acid positions in SEQ ID NO: 1 has or consists of an amino acid sequence of SEQ ID NO: 14. In some embodiments, the polypeptide having the modifications of S54C and A515C amino acid substitutions has a disulfide bond formed connecting position C54 and position C515 based on amino acid positions in SEQ ID NO: 1 to stabilize a prefusion conformation of the polypeptide.

In some embodiments, at least two cysteine amino acid residues can be substituted at position S55 and at position G227 based on amino acid positions in SEQ ID NO: 1. In some embodiments, the substitutions with the one or more pairs of engineered sulfhydryl-containing amino acid residues comprise amino acid substitutions of S55C and G227C compared to SEQ ID NO: 1 based on amino acid positions in SEQ ID NO: 1. In some embodiments, the polypeptide having the modifications of S55C and G227C amino acid substitutions based on amino acid positions in SEQ ID NO: 1 has or consists of an amino acid sequence of SEQ ID NO: 3. In some embodiments, the polypeptide having the modifications of S55C and G227C amino acid substitutions has a disulfide bond formed connecting position C55 and position C227 based on amino acid positions in SEQ ID NO: 1 to stabilize a prefusion conformation of the polypeptide.

In still other embodiments, at least two cysteine amino acid residues can be substituted at position H56 and at position G227 based on amino acid positions in SEQ ID NO: 1. In some embodiments, the substitutions with the one or more pairs of engineered sulfhydryl-containing amino acid residues comprise amino acid substitutions of H56C and G227C compared to SEQ ID NO: 1 based on amino acid positions in SEQ ID NO:1. In some embodiments, the polypeptide having the modifications of H56C and G227C amino acid substitutions based on amino acid positions in SEQ ID NO: 1 has or consists of an amino acid sequence of SEQ ID NO: 4. In some embodiments, the polypeptide having the modifications of S55C and G227C amino acid substitutions has a disulfide bond formed connecting position C56 and position C227 based on amino acid positions in SEQ ID NO: 1 to stabilize a prefusion conformation of the polypeptide.

In some embodiments, at least two cysteine amino acid residues can be substituted at position A175 and at position V529 based on amino acid positions in SEQ ID NO: 1. In some embodiments, the substitutions with the one or more pairs of engineered sulfhydryl-containing amino acid residues comprise amino acid substitutions of A175C and V529C compared to SEQ ID NO: 1 based on amino acid positions in SEQ ID NO: 1. In some embodiments, the polypeptide having the modifications of A175C and V529C amino acid substitutions based on amino acid positions in SEQ ID NO: 1 has or consists of an amino acid sequence of SEQ ID NO: 5. In some embodiments, the polypeptide having the modifications of A175C and V529C amino acid substitutions based on amino acid positions in SEQ ID NO: 1 has or consists of an amino acid sequence of SEQ ID NO: 15. In some embodiments, the polypeptide having the modifications of A175C and V529C amino acid substitutions has a disulfide bond formed connecting position C175 and position C529 based on amino acid positions in SEQ ID NO: 1 to stabilize a prefusion conformation of the polypeptide.

In other embodiments, at least two cysteine amino acid residues can be substituted at position G322 and at position A480 based on amino acid positions in SEQ ID NO: 1. In some embodiments, the substitutions with the one or more pairs of engineered sulfhydryl-containing amino acid residues comprise amino acid substitutions of G322C and A480C compared to SEQ ID NO:1 based on amino acid positions in SEQ ID NO:1. In some embodiments, the polypeptide having the modifications of G322C and A480C amino acid substitutions based on amino acid positions in SEQ ID NO: 1 has or consists of an amino acid sequence of SEQ ID NO: 6. In some embodiments, the polypeptide having the modifications of G322C and A480C amino acid substitutions based on amino acid positions in SEQ ID NO: 1 has or consists of an amino acid sequence of SEQ ID NO: 16. In some embodiments, the polypeptide having the modifications of G322C and A480C amino acid substitutions has a disulfide bond formed connecting position C322 and position C480 to stabilize a prefusion conformation of the polypeptide.

In still other embodiments, at least two cysteine amino acid residues can be substituted at position S325 and at position A480 based on amino acid positions in SEQ ID NO: 1. In some embodiments, the substitutions with the one or more pairs of engineered sulfhydryl-containing amino acid residues comprise amino acid substitutions of S325C and A480C compared to SEQ ID NO: 1 based on amino acid positions in SEQ ID NO:1. In some embodiments, the polypeptide having the modifications of S325C and A480C amino acid substitutions based on amino acid positions in SEQ ID NO: 1 has or consists of an amino acid sequence of SEQ ID NO: 7. In some embodiments, the polypeptide having the modifications of S325C and A480C amino acid substitutions based on amino acid positions in SEQ ID NO: 1 has or consists of an amino acid sequence of SEQ ID NO: 17. In some embodiments, the polypeptide having the modifications of S325C and A480C amino acid substitutions has a disulfide bond formed connecting position C325 and position C480 to stabilize a prefusion conformation of the polypeptide.

To facilitate prefusion stabilization in various embodiments, at least two cysteine amino acid residues can be substituted at position G322 and at position D478 based on amino acid positions in SEQ ID NO:1. In some embodiments, the substitutions with the one or more pairs of engineered sulfhydryl-containing amino acid residues comprise amino acid substitutions of G322C and D478C compared to SEQ ID NO:1 based on amino acid positions in SEQ ID NO:1. In some embodiments, the polypeptide having the modifications of G322C and D478C amino acid substitutions based on amino acid positions in SEQ ID NO: 1 has or consists of an amino acid sequence of SEQ ID NO: 8. In some embodiments, the polypeptide having the modifications of G322C and D478C amino acid substitutions based on amino acid positions in SEQ ID NO: 1 has or consists of an amino acid sequence of SEQ ID NO: 18. In some embodiments, the polypeptide having the modifications of G322C and D478C amino acid substitutions has a disulfide bond formed connecting position C322 and position C478 to stabilize a prefusion conformation of the polypeptide.

In still other embodiments, at least two cysteine amino acid residues can be substituted at position L696 and at position S727 based on amino acid positions in SEQ ID NO: 1. In some embodiments, the substitutions with the one or more pairs of engineered sulfhydryl-containing amino acid residues comprise amino acid substitutions of L696C and S727C compared to SEQ ID NO: 1 based on amino acid positions in SEQ ID NO:1. In some embodiments, the polypeptide having the modifications of L696C and S727C amino acid substitutions based on amino acid positions in SEQ ID NO: 1 has or consists of an amino acid sequence of SEQ ID NO: 9. In some embodiments, the polypeptide having the modifications of L696C and S727C amino acid substitutions has a disulfide bond formed connecting position C696 and position C727 to stabilize a prefusion conformation of the polypeptide.

In some embodiments, at least two cysteine amino acid residues can be substituted at position A175 and at position V178 based on amino acid positions in SEQ ID NO: 1. In some embodiments, the substitutions with the one or more pairs of engineered sulfhydryl-containing amino acid residues comprise amino acid substitutions of A175C and V178C compared to SEQ ID NO: 1 based on amino acid positions in SEQ ID NO:1. In some embodiments, the polypeptide having the modifications of A175C and V178C amino acid substitutions based on amino acid positions in SEQ ID NO: 1 has or consists of an amino acid sequence of SEQ ID NO: 10. In some embodiments, the polypeptide having the modifications of A175C and V178C amino acid substitutions based on amino acid positions in SEQ ID NO:1 has or consists of an amino acid sequence of SEQ ID NO: 19. In some embodiments, the polypeptide having the modifications of A175C and V178C amino acid substitutions has a disulfide bond formed connecting position C175 and position C178 to stabilize a prefusion conformation of the polypeptide.

In other embodiments, at least two cysteine amino acid residues can be substituted at position G172 and at position D564 based on amino acid positions in SEQ ID NO: 1. In some embodiments, the substitutions with the one or more pairs of engineered sulfhydryl-containing amino acid residues comprise amino acid substitutions of G172C and D564C compared to SEQ ID NO: 1 based on amino acid positions in SEQ ID NO:1. In some embodiments, the polypeptide having the modifications of G172C and D564C amino acid substitutions based on amino acid positions in SEQ ID NO: 1 has or consists of an amino acid sequence of SEQ ID NO: 11. In some embodiments, the polypeptide having the modifications of G172C and D564C amino acid substitutions based on amino acid positions in SEQ ID NO:1 has or consists of an amino acid sequence of SEQ ID NO: 20. In some embodiments, the polypeptide having the modifications of G172C and D564C amino acid substitutions has a disulfide bond formed connecting position C172 and position C564 to stabilize a prefusion conformation of the polypeptide.

In still other embodiments, at least two cysteine amino acid residues can be substituted at position L580 and at position Y644 based on amino acid positions in SEQ ID NO: 1. In some embodiments, the substitutions with the one or more pairs of engineered sulfhydryl-containing amino acid residues comprise amino acid substitutions of L580C and Y644C compared to SEQ ID NO: 1 based on amino acid positions in SEQ ID NO:1. In some embodiments, the polypeptide having the modifications of L580C and Y644C amino acid substitutions based on amino acid positions in SEQ ID NO: 1 has or consists of an amino acid sequence of SEQ ID NO: 12. In some embodiments, the polypeptide having the modifications of L580C and Y644C amino acid substitutions based on amino acid positions in SEQ ID NO:1 has or consists of an amino acid sequence of SEQ ID NO: 21. In some embodiments, the polypeptide having the modifications of L580C and Y644C amino acid substitutions has a disulfide bond formed connecting position C580 and position C644 to stabilize a prefusion conformation of the polypeptide.

Alternatively, the substitutions with sulfhydryl-containing, or cysteine, amino acid residues in the polypeptides disclosed herein can also be designated based on amino acid positions in SEQ ID NO:27. Since the sequence of SEQ ID NO:27 aligns with residues 23-683 of SEQ ID NO:1, the amino acid substitution based on positions in SEQ ID NO:27 are positioned with a 22-residue difference compared to the positions in SEQ ID NO:1. For example, amino acid substitutions S54C, S55C, H56C, G172C, A175C, V178C, G227C, G322C, S325C, D478C, A480C, A515C, V529C, D564C, L580C, Y644C, L696C, and S727C based on amino acid positions in SEQ ID NO:1 are S32C, S33C, H34C, G150C, A153C, V156C, G205C, G300C, S303C, D456C, A458C, A493C, V507C, D542C, L558C, Y622C, L674C, and S705C, respectively, based on amino acid positions in SEQ ID NO:27.

Additional embodiments provide that the modifications include substitutions with two or more pairs of sulfhydryl-containing, or cysteine, amino acid residues. In various aspects, a polypeptide is provided, which contains an altered EBV gB ectodomain sequence that has modifications relative to the native EBV gB ectodomain sequence, wherein the modifications include or are substituting two, three, or more pairs of amino acid residues with two, three, or more pairs, respectively of engineered sulfhydryl-containing amino acid residues. In some aspects, a polypeptide is provided, which contains an altered EBV gB ectodomain sequence that has modifications relative to the native EBV gB ectodomain sequence, wherein the modifications include or are substituting two pairs of amino acid residues with two pairs of engineered sulfhydryl-containing amino acid residues. In some aspects, a polypeptide is provided, which contains an altered EBV gB ectodomain sequence that has modifications relative to the native EBV gB ectodomain sequence, wherein the modifications include or are substituting two pairs of amino acid residues with two pairs of engineered sulfhydryl-containing amino acid residues.

In some embodiments, the substitutions with two or more pairs of cysteine amino acid residues are selected from any two, three, four, five, six, seven, eight, nine, ten, or all pairs of S54C and A515C, S55C and G227C, H56C and G227C, A175C and V529C, G322C and A480C, S325C and A480C, G322C and D478C, L696C and S727C, A175C and V178C, G172C and D564C, and L580C and Y644C, wherein the amino acid numbering is based on amino acid positions in SEQ ID NO:1.

In some embodiments, the substitutions with two or more pairs of cysteine amino acid residues are selected from any two pairs of A175C and V529C, G322C and A480C, and G172C and D564C, wherein the amino acid numbering is based on amino acid positions in SEQ ID NO:1. In some embodiments, the substitutions with two or more pairs of cysteine amino acid residues are A175C and V529C, and G322C and A480C, wherein the amino acid numbering is based on amino acid positions in SEQ ID NO:1. In some embodiments, the substitutions with two or more pairs of cysteine amino acid residues are A175C and V529C, and G172C and D564C, wherein the amino acid numbering is based on amino acid positions in SEQ ID NO:1. In some embodiments, the substitutions with two or more pairs of cysteine amino acid residues are G322C and A480C, and G172C and D564C, wherein the amino acid numbering is based on amino acid positions in SEQ ID NO:1. In some embodiments, the substitutions with two or more pairs of cysteine amino acid residues are three pairs of cysteine amino acid residues, A175C and V529C, G322C and A480C, and G172C and D564C, wherein the amino acid numbering is based on amino acid positions in SEQ ID NO:1.

In various embodiments, the modifications of the altered EBV gB ectodomain sequence of the polypeptide, relative to the native EBV gB ectodomain sequence, exclude amino acid substitutions of (1) R49C, while wild type residue C528 remains unchanged; (2) A515C, while wild type residue C51 remains unchanged; (3) L82C and T306C; (4) Y99C and V127C; (5) L107C and N119C; (6) N110C and P191C; (7) N143C and D158C; (8) A144C and N163C; (9) D158C and V204C; (10) L168C and Y181C; (11) S183C, while wild type residue C206 remains unchanged; (12) M211C and T225C; (13) S215C and F222C; (14) E231C and H245C; (15) S233C and E242C; (16) Y236C and E242C; (17) R247C and S250C; (18) G365C and P384C; (19) T374C and L378C; (20) A418C and R419C; (21) R419C and G420C; (22) G420C and S421C; (23) M506C and A516C; (24) T535C and S560C; (25) R539C and L556C; (26) K540C and H610C; (27) S547C and M550C; and (28) Q589C and S592C, wherein all amino acid numbering in (1)-(28) is based on amino acid positions in SEQ ID NO:1. In various embodiments, the modifications of the altered EBV gB ectodomain sequence of the polypeptide, relative to the native EBV gB ectodomain sequence, further include substituting one or both native cysteine amino acid residues with another amino acid residues that does not contain a sulfhydryl group in (i) C51 and C528; (ii) C68 and C484; (iii) C141 and C206; (iv) C295 and C342; and (v) C551 and C588, wherein all amino acid number in (i)-(v) is based on amino acid positions in SEQ ID NO:1.

In various embodiments, the modifications of the altered EBV gB ectodomain sequence of the polypeptide, relative to the native EBV gB ectodomain sequence, exclude the amino acid substitutions of (1) R49C, while wild type residue C528 remains unchanged; (2) A515C, while wild type residue C51 remains unchanged; (3) L82C and T306C; (4) Y99C and V127C; (5) L107C and N119C; (6) N110C and P191C; (7) N143C and D158C; (8) A144C and N163C; (9) D158C and V204C; (10) L168C and Y181C; (11) S183C, while wild type residue C206 remains unchanged; (12) M211C and T225C; (13) S215C and F222C; (14) E231C and H245C; (15) S233C and E242C; (16) Y236C and E242C; (17) R247C and S250C; (18) G365C and P384C; (19) T374C and L378C; (20) A418C and R419C; (21) R419C and G420C; (22) G420C and S421C; (23) M506C and A516C; (24) T535C and S560C; (25) R539C and L556C; (26) K540C and H610C; (27) S547C and M550C; and (28) Q589C and S592C; include substituting one or both native cysteine amino acid residues with an amino acid residue selected from Ala, Arg, As, Asp, Glu, Gln, Gly, His, Ile, Leu, Lys, Met, Phe, Pro, Ser, Thr, Trp, Tyr, and Val in the pairs of (i) C51 and C528; (ii) C68 and C484; (iii) C141 and C206; (iv) C295 and C342; and (v) C551 and C588; and include one or two, or three, or more pairs of amino acid substitutions selected from S54C and A515C, S55C and G227C, H56C and G227C, A175C and V529C, G322C and A480C, S325C and A480C, G322C and D478C, L696C and S727C, A175C and V178C, G172C and D564C, and L580C and Y644C, wherein the amino acid numbering is based on amino acid positions in SEQ ID NO:1.

In some embodiments, an agent is used to form the disulfide bond between the one or more pairs of cysteine amino acid residues. An oxidizing agent, e.g., H2O2, glutathione, L-cysteine, cysteamine, is suitable for use in inducing formation of disulfide bonds.

In various embodiments, an altered EBV gB ectodomain sequence is otherwise identical to the native EBV gB ectodomain sequence except for those modifications, i.e., the unmodified fragments share 100% sequence identity to the native sequence. In other embodiments, an altered EBV gB ectodomain sequence is otherwise a conservative variant to the native EBV gB ectodomain sequence except for those modifications.

Additional embodiments provide that the modifications of the altered EBV gB ectodomain sequence relative to the native EBV gB ectodomain sequence further include amino acid substitutions at one or more positions selected from W112, Y113, W193, L194, 1195, and W196 based on amino acid positions in SEQ ID NO:1. In some embodiments, the modifications are wherein wild type residues WY¹¹²⁻¹¹³ and WLIW¹⁹³⁻¹⁹⁶ (SEQ ID NO:28) based on positions in SEQ ID NO:1 are replaced with HR and RVEA (SEQ ID NO:29) in the altered EBV gB ectodomain sequence.

In some embodiments, the polypeptide, e.g., in the altered EBV gB ectodomain sequence, can further comprise an additional modification, wherein the additional modification comprises substitution of at least one native cysteine amino acid residue with another amino acid residue selected from the group of Ala, Arg, As, Asp, Glu, Gln, Gly, His, Ile, Leu, Lys, Met, Phe, Pro, Ser, Thr, Trp, Tyr, and Val. In other embodiments, the polypeptide, e.g., in the altered EBV gB ectodomain sequence, can further comprise one or more additional modifications. In other embodiments, the additional modification can be substituting one or more proline amino acid residues at a position selected from one of R515, H516, and V517, wherein the amino acid numbering is based on amino acid position in SEQ ID NO: 1. In still other embodiments, the additional modification can include a C-terminal truncation. In still other embodiments, the additional modification can include addition of a C-terminal scaffold. In some embodiments, the additional modification can include addition of a trimerization domain. In some embodiments, the trimerization domain can include a T4 fibritin trimerization domain. In some embodiments, the polypeptide has one or more N-glycosylated Asn residues in the ectodomain; or that is, one or more N-glycosylated Asn residues in the altered EBV gB ectodomain sequence. In some aspects, the polypeptide is modified by O-linked carbohydrates in one or more residues (such as Pro, Ser, and Thr residues). In some aspects, the engineered polypeptide does not have the hydrophobic, ˜40-residues long, stem region or the transmembrane domain. While wildtype EBV gB ectodomain extends into the fairly hydrophobic stem region and the transmembrane domain, removing the stem region and the transmembrane domain (due to their hydrophobicity) helps with expression of the recombinantly engineered gB.

In various embodiments, a polypeptide disclosed herein further contains, in addition to the altered gB ectodomain sequence, one or more of:
(i) a signal sequence, preferably on the N-terminus end relative to the altered gB ectodomain sequence,
(ii) a marker sequence,
(iii) a trimerization domain, preferably on the C-terminus end relative to the altered gB ectodomain sequence,
(iv) a poly histidine affinity tag, and
(v) one or more linker sequences.

In some embodiments, the polypeptide is in a configuration from the N-terminal to C-terminal direction: signal sequence-marker sequence-altered gB ectodomain sequence-linker sequence 1-trimerization domain-linker sequence 2-poly histidine affinity tag. In other embodiments, the polypeptide is in a configuration from the N-terminal to C-terminal direction: signal sequence-marker sequence-altered gB ectodomain sequence-linker sequence 1-trimerization domain-poly histidine affinity tag. In yet another embodiment, the polypeptide is in a configuration from the N-terminal to C-terminal direction: signal sequence-altered gB ectodomain sequence-linker sequence 1-trimerization domain-linker sequence 2-poly histidine affinity tag. In some embodiments, linker sequence 1 and linker sequence 2 have identical sequence. For example, a linker sequence can be a two amino acid sequence Gly-Ser. In some embodiments, linker sequence 1 and linker sequence 2 have different sequences. In some embodiments, linker sequences are flexible linker sequences. Alternative linker sequences can be GGGGS (SEQ ID NO:49) (also referred to as GS linker) or multiple repeats (n=2, 3, 4, 5, 6, 7, 8, or more) thereof.

Signal sequences, or signal peptides or leader peptide sequences, may be included in the polypeptides, to improve transgene expression/secretion. Generally, they are sequences of 16 to 20 amino acids at the N-terminus of eukaryotic proteins directing newly synthesized proteins, such as during recombinant production of the polypeptides disclosed herein, toward the secretory pathway. Generally, the signal sequence for a protein destined to enter the endoplasmic reticulum always contains hydrophobic amino acids that become embedded in the lipid bilayer membrane, and it functions to guide the nascent protein to a receptor protein that marks the position of a pore in the membrane. Once the protein passes into the cysternal lumen through the pore, the leader segment is cleaved from the protein.

In some embodiments, the signal peptide comprises the amino acid sequence derived from the murine immunoglobulin kappa (IgGκ) light chain, or METDTLLLWVLLLWVPGSTG (SEQ ID NO:31). In some aspects, cleavage of a signal peptide comprising the amino acid sequence of SEQ ID NO:31 occurs on the C-terminal end relative to the last G residue. In exemplary sequences of SEQ ID NOs:13-23 and 25-28, a short FLAG tag (as a marker sequence) is included on the C-terminal end relative to the signal sequence to ensure the signal sequence is cleaved predictably. In some embodiments, other signal sequences or native signal sequence of the EBV gB can be included in place of the IgG kappa light chain in the polypeptide, without the marker sequence or short FLAG tag. Additional signal sequences, suitable for use in mammalian cell expression systems, include but are not limited to human OSM (MGVLLTQRTLLSLVLALLFPSMASM (SEQ ID NO: 33)), VSV-G (MKCLLYLAFLFIGVNC (SEQ ID NO:34)), mouse Ig Heavy (MGWSCIILFL VATATGVHS (SEQ ID NO:35)), BM40 (MRAWIFFLLCLAGRALA (SEQ ID NO: 36)), Secrecon (MWWRLWWLLLLLLLLWPMVWA (SEQ ID NO:37)), human IgKVIII (MDMRVPAQLLGLLLLWLRGARC (SEQ ID NO:38)), CD33 (MPLLLLLPLLWAGALA (SEQ ID NO:39)), tPA (MDAMKRGLCCVLLLCGAVFVSPS (SEQ ID NO:40)), human chymotrypsinogen (MAFLWLLSCWALLGTTFG (SEQ ID NO: 41)), human trypsinogen-2 (MNLLLILTFVAAAVA (SEQ ID NO:42)), human IL-2 (MYRMQLLSCIALSLALVTNS (SEQ ID NO:43)), Gaussia luc (MGVKVLFALICIAVAEA (SEQ ID NO:44)), albumin (HSA) (MKWVTFISLLFSSAYS (SEQ ID NO:45)), Influenza Haemagglutinin (MKTIIALSYIFCLVLG (SEQ ID NO:46)), Human insulin (MALWMRLLPLLALLALWGPDPAAA (SEQ ID NO:47)), and Silkworm Fibroin LC (MKPIFLVLLVVTSAYA (SEQ ID NO:48)). Examples of signal sequences that perform well in gram-positive bacteria are described in Front Bioeng Biotechnol. 2019; 7:139.

In some embodiments, the polypeptide disclosed herein includes a multimerization domain. Preferably, the multimerization domain is a trimerization domain. Preferably the trimerization domain is on the C-terminus end relative to the altered gB ectodomain sequence. In some embodiments, the trimerization domain includes or is T4 bacteriophage fibritin foldon (Fd) trimerization domain, which is at the C-terminal domain of T4 fibritin. In some embodiments, the trimerization domain comprises the amino acid sequence of GYIPEAPRDGQAYVRKDGEWVLLSTFL (SEQ ID NO:32). Generally, the T4 fibritin foldon consists of three β-hairpins, which assemble into a β-propeller-like structure, and the Fd trimer is stabilized by hydrogen-bonding, hydrophobic interactions, and salt-bridges between each. In other embodiments, the multimerization domain includes or is the isoleucine zipper based on the GCN4 transcriptional activator from Saccharomyces cerevisae. In some embodiments, the trimerization domain comprises an isoleucine zipper sequence comprising the amino acid sequence of RMKQIEDKIEEILSKIYHIENEIARIKKLIGER (SEQ ID NO:50). In some embodiments, the trimerization domain comprises a collagen triple helix sequence comprising the amino acid sequence of GSSGVRLWATRQAMLGQVHEVPEGWLIFVAEQEELYVRVQNGFRKVQLEARTPLP R (SEQ ID NO:51). In some embodiments, the trimerization domain comprises the amino acid sequence of EEAELAYLLGELAYKLGEYRIAIRAYRIALKRDPNNAEAWYNLGNAYYKQGDYDEA IEYYQKALELDPNNAEAWYNLGNAYYKQGDYDEAIEYYQKALELDPNNAEAKQNL GNAKQKQG (SEQ ID NO:52), which is a computationally designed C3-symmetric homotrimer from TPR repeat protein. In some embodiments, the trimerization domain comprises the amino acid sequence of XRMKQLEDKVEELLSKNYHLENEVARLKKLVGERX (SEQ ID NO:53), which is a GCN4 leucine zipper sequence. In some embodiments, the polypeptide further includes a multimerization domain comprising the amino acid sequence of MGSSHHHHHHSQDPMLSKDIIKLLNEQVNKEMNSSNLYMSMSSWCYTHSLDGAGL FLFDHAAEEYEHAKKLIIFLNENNVPVQLTSISAPEHKFEGLTQIFQKAYEHEQHISESI NNIVDHAIKSKDHATFNFLQWYVAEQHEEEVLFKDILDKIELIGNENHGLYLADQYV KGIAKSRKS (SEQ ID NO:54), which is derived from ferritin. For example, inclusion of a trimerization domain allows for the polypeptide to form a trimer (e.g., upon expression), and inclusion of the ferritin sequence helps display eight copies of trimerized polypeptides, thereby increasing multimerization. In additional embodiments, the polypeptide further includes a SpyCatcher, wherein the trimerization domain (motif) is fused to the SpyCatcher, to promote multimerization when it interacts with SpyTag on a virus-like-particle. SpyCatcher and SpyTag are described in Tan et al., Nature Communications volume 12, Article number: 542 (2021).

In some embodiments, the polypeptide disclosed herein includes a marker sequence. In some embodiments, the polypeptide disclosed herein does not have a marker sequence. In various aspects, the inclusion of a marker sequence facilitates/ensures post-translational cleavage of a signal sequence from the rest of the polypeptide. Preferably, the marker sequence is a short sequence, positioned between the signal sequence and the altered ectoS sequence. In some aspects, the marker sequence is a FLAG®-tag sequence. In some aspects, the marker sequence comprises the amino acid sequence of DYKDE (SEQ ID NO: 30).

In some aspects, the polypeptides disclosed herein are provided in a pharmaceutical composition. In some aspects, the polypeptides are formulated in a suspension or solution. In other aspects, the polypeptides are lyophilized.

Various embodiments, a protein is provided comprising three polypeptides as disclosed herein. In various aspects, a protein is provided comprising three copies of a polypeptide disclosed herein. In various aspects, a protein is provided comprising multiple copies of a polypeptide in a multimer conformation, wherein the polypeptide comprises a multimerization domain. In some aspects, the protein further includes an additional polypeptide. In some aspects, the protein further includes an additional glycoprotein, preferably a glycoprotein from EBV other than gB. In some aspects, the additional glycoprotein is EBV gHgL, gp42, gp350, gp220, complex gp350/220, or a combination thereof.

Also provided are isolated immunogens. In various embodiments, an isolated immunogen comprises a polypeptide disclosed herein, i.e., a polypeptide derived from the EBV gB, which includes an altered EBV gB ectodomain sequence that has modifications relative to the native EBV gB ectodomain sequence, said native gB ectodomain comprising the amino acid sequence set forth as residues 23-683 of SEQ ID NO:1 (or individually listed in SEQ ID NO:27), wherein the modifications comprise substituting one or more amino acid residues with one or more engineered sulfhydryl-containing amino acid residues.

Preferably, the modifications of the altered EBV gB ectodomain sequence in the polypeptide of the isolated immunogen relative to the native EBV gB ectodomain sequence include substituting one, two, three, or more pairs of amino acid residues with one, two, three, or more pairs of cysteine amino acid residues selected from S54C and A515C, S55C and G227C, H56C and G227C, A175C and V529C, G322C and A480C, S325C and A480C, G322C and D478C, L696C and S727C, A175C and V178C, G172C and D564C, and L580C and Y644C, wherein the amino acid numbering is based on amino acid positions in SEQ ID NO:1.

Additional embodiments provide immunogenic compositions to induce an immune response to a target Epstein-Barr virus (EBV) protein. In some aspects, the immunogenic composition is for use in inducing an immune response in a recipient of the immunogenic composition to EBV glycoprotein B (gB). In various embodiments, an immunogenic composition contains a first component comprising at least one construct encoding a polypeptide disclosed herein comprising an altered EBV gB ectodomain sequence including at least two engineered cysteine amino acid residues relative to the native EBV gB ectodomain sequence; and a second component comprising a pharmaceutically acceptable carrier. In some aspects, the cysteine amino acid residues are substituted for any amino acid residue(s) in the native EBV gB ectodomain sequence. In further aspects, at least one disulfide bond forms between the at least two cysteine residues to stabilize a prefusion conformation of the polypeptide.

Additional embodiments provide one or more nucleic acid molecules which encode a polypeptide derived from the EBV gB as disclosed herein. In various embodiments, a nucleic acid molecule is provided, which encodes a polypeptide comprising an altered gB ectodomain sequence that has modifications relative to native gB ectodomain sequence of the EBV, said native gB ectodomain comprising the amino acid sequence set forth as residues 23-683 of SEQ ID NO:1, wherein the modifications comprise substituting one or more pairs of amino acid residues with one or more pairs of engineered sulfhydryl-containing amino acid residues. In some embodiments, a nucleic acid molecule is provided, which encodes a polypeptide comprising an altered gB ectodomain sequence that has modifications relative to the native gB ectodomain sequence of the EBV, wherein the modifications comprise substituting one or more pairs of amino acid residues selected from S54C and A515C, S55C and G227C, H56C and G227C, A175C and V529C, G322C and A480C, S325C and A480C, G322C and D478C, L696C and S727C, A175C and V178C, G172C and D564C, and L580C and Y644C, wherein the amino acid number is based on amino acid positions in SEQ ID NO: 1. In some embodiments, the nucleic acid molecule is provided, which encodes the polypeptide comprising the altered EBV gB ectodomain sequence and a trimerization domain at the C-terminus end relative to the altered EBV gB ectodomain sequence. In some embodiments, the nucleic acid molecule is provided, which encodes the polypeptide comprising the altered EBV gB ectodomain sequence and a signal sequence at the N-terminus end relative to the altered EBV gB ectodomain sequence. In some embodiments, the nucleic acid molecule is provided, which encodes the polypeptide comprising the altered EBV gB ectodomain sequence, a trimerization domain at the C-terminus end relative to the altered EBV gB ectodomain sequence, and a signal sequence at the N-terminus end relative to the altered EBV gB ectodomain sequence. In some embodiments, a nucleic acid molecule is provided which encodes a polypeptide comprising the amino acid sequence as set forth in any one of SEQ ID NOs: 1-12 and 14-26.

Preferably, the nucleic acid molecules are ribonucleic acid (RNA), for use as an immunogenic composition. RNA (e.g., messenger RNA (mRNA)) can safely direct the body's cellular machinery to produce nearly any protein of interest. The RNA (e.g., mRNA) vaccines have superior properties in that they can produce much larger antibody titers and produce responses earlier than commercially available anti-viral therapeutic treatments.

More preferably, the nucleic acid molecules are a messenger ribonucleic acid (mRNA) polynucleotide. mRNA polynucleotides are conceived to produce the appropriate protein conformation upon translation. In various aspects the mRNA comprises an open reading frame encoding a polypeptide derived from the EBV gB as disclosed herein. In various aspects the mRNA comprises a 5′ untranslated region (UTR), an open reading frame encoding the polypeptide derived from the EBV gB, a 3′ UTR, and a poly (A) tail. In some aspects, the mRNA further comprises a 5′ cap analog. In some aspects, one or more or majority (e.g., 50%, 60%, 70%, 80% or greater) of uracil in the open reading frame has a chemical modification, such as 1-methylpseudouridine modification or a 1-ethylpseudouridine modification.

In various embodiments, the mRNA is formulated in a lipid nanoparticle, so as to form an immunogenic composition. In various aspects, the lipid nanoparticle comprises an ionizable cationic lipid, a neutral lipid, a sterol, and a PEG-modified lipid. In some aspects, he lipid nanoparticle comprises 20-60% ionizable cationic lipid, 5-25% neutral lipid, 25-55% cholesterol, and 0.5-15% PEG-modified lipid. In some aspects, the lipid nanoparticle comprises 50% ionizable cationic lipid, 10% neutral lipid, 38.5% sterol, and 1.5% PEG-modified lipid. In some aspects, neutral lipid is 1,2-distearoyl-sn-glycero-3-phosphocholine (DSPC), the sterol is cholesterol, and the PEG-modified lipid is 1,2-dimyristoyl-racalycero-3-methoxypolyethylene glycol-2000 (PEG-DMG) or PEG-CDMA. Additional description of the components in mRNA-based immunogenic compositions is provided in U.S. Pat. No. 10,702,600, which is incorporated by reference herein.

### Methods of Uses

Additional embodiments of the disclosure provide methods to induce an immunogenic response in a mammalian subject. Further embodiments provide the methods are for inducing an immunogenic response to a target EBV protein in a mammalian subject. Further embodiments provide the methods are for inducing an immunogenic response to EBV in a mammalian subject. In some embodiments, an immunogenic response includes generating neutralizing antibodies against the target EBV protein. In some embodiments, the target EBV protein can be an EBV glycoprotein B (gB). In some embodiments, an immunogenic response is a protective immune response.

In some embodiments, the methods to induce an immunogenic response in a mammalian subject include administering a polypeptide disclosed herein derived from the EBV gB to a subject in need thereof. That is, in some embodiments, a method is provided to induce an immunogenic response to the EBV gB in a mammalian subject, preferably human, which includes administering a polypeptide derived from the EBV gB, which includes an altered EBV gB ectodomain sequence that has modifications relative to the native EBV gB ectodomain sequence, said native gB ectodomain comprising the amino acid sequence set forth as residues 23-683 of SEQ ID NO:1 (or individually listed in SEQ ID NO:27), wherein the modifications comprise substituting one or more amino acid residues with one or more engineered sulfhydryl-containing amino acid residues. In some embodiments, the polypeptide for use in inducing an immunogenic response in a mammalian subject does not include a signal sequence at the N-terminus end relative to the altered EBV gB ectodomain sequence. In some embodiments, the polypeptide for use in inducing an immunogenic response in a mammalian subject is a post-translational recombinant polypeptide encoded by a nucleic acid construct encoding the polypeptide disclosed herein.

In some embodiments, a method to induce an immunogenic response in a mammalian subject includes administering an immunogenic composition comprising a first component comprising at least one construct encoding a polypeptide disclosed herein derived from the EBV gB, and a second component comprising a pharmaceutically acceptable carrier.

In some embodiments, a method to induce an immunogenic response in a mammalian subject includes administering at least one construct encoding a polypeptide disclosed herein derived from the EBV gB. In further embodiments, the construct encoding the polypeptide derived from the EBV gB is an mRNA. In various aspects, a method to induce an immunogenic response in a mammalian subject includes administering at least an mRNA having an open reading frame encoding the polypeptide derived from the EBV gB.

In some embodiments, a method to induce an immunogenic response in a mammalian subject includes administering co-administering two immunogenic components, a first component comprising at least one construct encoding a first polypeptide disclosed herein derived from the EBV gB; and a second component comprising a second polypeptide disclosed herein derived from the EBV gB, wherein the two components are administered to a subject at a first anatomical site. In some embodiments, the administration of the two components simultaneously, enhances the immune response compared to administering the first component in the absence of the second component. In some embodiments, the first polypeptide encoded by the construct in the first component and the second polypeptide in the second component are the same polypeptide. In other embodiments, the first polypeptide encoded by the construct in the first component and the second polypeptide in the second component are polypeptides having different amino acid substitutions relative to the native EBV gB ectodomain sequence.

In some embodiments, a method to induce an immunogenic response in a mammalian subject includes administering the immunogenic composition which further comprises a third component comprising a polypeptide, wherein the polypeptide is a polypeptide disclosed herein derived from the EBV gB. In some embodiments, the immunogenic composition can comprise a first component that can encode a polypeptide with a sequence that can match the polypeptide used in the third component. In some embodiments, the immunogenic composition can comprise a first component that can encode a polypeptide with a sequence that does not match the polypeptide used in the third component.

In still other embodiments, a method to induce an immunogenic response in a mammalian subject includes administering an immunogenic composition, wherein the immunogenic composition includes a polypeptide disclosed herein derived from the EBV gB and/or a construct encoding the polypeptide, and at least one adjuvant.

Adjuvant is a vehicle used to enhance antigenicity. Exemplary adjuvants suitable for inclusion in the immunogenic compositions disclosed herein include a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages) Immunostimulatory oligonucleotides (such as those including a CpG motif) can also be used as adjuvants. Adjuvants include biological molecules (a “biological adjuvant”), such as costimulatory molecules. Other exemplary adjuvants include IL-2, RANTES, GM-CSF, TNF-α, IFN-γ, G-CSF, LFA-3, CD72, B7-1, B7-2, OX-40L, 4-1BBL and toll-like receptor (TLR) agonists, such as TLR-9 agonists. The person of ordinary skill in the art is familiar with adjuvants (see, e.g., Singh (ed.) Vaccine Adjuvants and Delivery Systems. Wiley-Interscience, 2007).

In still other embodiments, the method to induce an immunogenic response to a target EBV protein can comprise combining an immunogenic composition disclosed herein with at least one additional immunogenic composition directed to at least one target protein specific to at least one additional virus.

In various aspects of the methods for inducing an immunogenic response, the polypeptide in the immunogenic composition, or the polypeptide encoded by a construct in the immunogenic composition, has modifications relative to native EBV gB ectodomain that include substituting at least one pair of amino acid residues with one pairs of cysteine amino acid residues selected from S54C and A515C, S55C and G227C, H56C and G227C, A175C and V529C, G322C and A480C, S325C and A480C, G322C and D478C, L696C and S727C, A175C and V178C, G172C and D564C, and L580C and Y644C, wherein the amino acid number is based on amino acid positions in SEQ ID NO:1. In some embodiments, the modifications include substituting two or three pairs of amino acid residues with two or three pairs of cysteine amino acid residues selected from S54C and A515C, S55C and G227C, H56C and G227C, A175C and V529C, G322C and A480C, S325C and A480C, G322C and D478C, L696C and S727C, A175C and V178C, G172C and D564C, and L580C and Y644C, wherein the amino acid number is based on amino acid positions in SEQ ID NO:1. In some embodiments, the modifications include substituting three or more pairs of pairs of amino acid residues with three or more pairs of cysteine amino acid residues selected from S54C and A515C, S55C and G227C, H56C and G227C, A175C and V529C, G322C and A480C, S325C and A480C, G322C and D478C, L696C and S727C, A175C and V178C, G172C and D564C, and L580C and Y644C, wherein the amino acid number is based on amino acid positions in SEQ ID NO:1. Preferably, a disulfide bond forms between each pair of the at least two cysteine residues, so as to stabilize a prefusion conformation of the polypeptide.

In some aspects, the method of administering an immunogenic composition disclosed herein inhibits development of a disease or condition or reduces the likelihood of development of a disease or condition, for example, in a subject who is at risk for a disease such as EBV infection. In various embodiments, the disease or condition is mononucleosis.

In some embodiments, the method to induce an immunogenic response to a target EBV protein in a mammalian subject includes administering the immunogenic composition disclosed herein to the subject at least twice, e.g., a primer-boost vaccination. In some aspects, the method of inducing an immunogenic response includes inducing multiple exposures of the subject to the immunogenic compositions, wherein the exposures can have a time interval of 1 week, 2 weeks, 3 weeks, 4 weeks, 5 weeks, 6 weeks, 7 weeks, 8 weeks, 9 weeks, 10 weeks, 2 months, 3 months, 4 months, 5 months, 6 months, 7 months, 8 months, 9 months, 10 months, 11 months, 1 year, 1.5 years, 2 years, 3 years, 4 years, 5 years, 6 years, 7 years, 8 years, 9 years, or 10 years, or given at another frequency as determined by a healthcare professional.

In some embodiments, the method is to induce an immunogenic response in a subject in need thereof. A subject in need of generating an immunogenic response includes one that is immunologically naïve with respect to the target EBV protein. In other embodiments, the method to induce an immunogenic response to a target EBV protein can comprise a subject that has been previously exposed to EBV. In some embodiments, a subject in need thereof is a pediatric human. In some embodiments, a subject in need thereof is an infant (1 year old or younger). In some embodiments, a subject in need thereof is a young child, e.g., between 1 and 10 years old. In some embodiments, a subject in need thereof is a teenager. In various embodiments, the human subject is age 1 day through 5 month, 6 months through 4 years, 5 years through 11 years, or 12 years through 17 years.

### Methods of Preparing

In various embodiments, the polypeptides disclosed herein are recombinantly produced in an expression system of choice. In some embodiments, the polypeptides are produced in a mammalian expression system. In some embodiments, the polypeptides are produced in a transient expression system. In some embodiments, the polypeptides are produced with a lentiviral vector used to transduce cells resulting in stable transductants. In some embodiments, the polypeptides are produced in human cell lines using a lentiviral vector. In some embodiments, a Daedalus expression system, e.g., lentiviral transduction of serum-free adapted human 293 Freestyle (293-F) cells, can be used in the producing recombinant EBV gB variants as disclosed herein. Generally in the Daedalus expression system, the inclusion of minimized ubiquitous chromatin opening elements in the transduction vectors prevents genomic silencing and maintains the stability of decigram levels of expression.

A polynucleotide can be inserted into an expression vector that contains a promoter sequence, which facilitates the efficient transcription of the inserted genetic sequence of the host. The expression vector typically contains an origin of replication, a promoter, as well as specific nucleic acid sequences that allow phenotypic selection of the transformed cells.

While illustrative embodiments have been illustrated and described, it will be appreciated that various changes can be made therein without departing from the spirit and scope of the invention.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

We designed proof-of-concept prefusion stabilized EBV gB by computationally determining the placement of cysteine amino acids that would form stabilizing disulfide bonds. Several constructs of disulfide-stabilized variants of EBV gB included a fibritin foldon trimerization domain and His affinity tags at the c-terminus. Two sets of constructs were designed with one set containing the native fusion loop amino acid sequences (e.g., SEQ ID NO:22) and a second set containing fusion loop substitutions (e.g., SEQ ID NOs:14-21 and 23-26).

Our design strategy began with generating a predicted model of the metastable prefusion state of EBV gB using publicly available protein folding software program, Alphafold Multimer. We then computationally determined the placement of disulfide bonds to help tether domains which are close together in the prefusion state, but far apart in the postfusion state. Constructs containing a fibritin foldon trimerization domain facilitated trimerization of gB variants (or altered gB ectodomain sequence) and acted as a scaffold with the ability to present one copy of EBV gB variant as a trimer. Poly histidine affinity tags were included for ease of purification using Ni-NTA affinity. Lastly, fusion loops of EBV gB were substituted with HSV-1 fusion loop residues to reduce the formation of rosettes with one set. This process facilitated the design of several candidate cysteine variants (e.g., SEQ ID NOs:14-21) which are likely to stabilize the EBV gB prefusion state.

Eight disulfide-stabilized constructs were designed with fusion loop substitutions (e.g., SEQ ID NOs:14-21). Eight disulfide-stabilized constructs were designed with native fusion loop sequences (corresponding to those of SEQ ID NOs:14-21 but containing WY¹¹²⁻¹¹³ and WLIW¹⁹³⁻¹⁹⁶, wherein the amino acid numbering is based on amino acid positions in SEQ ID NO:1).

### Example 2

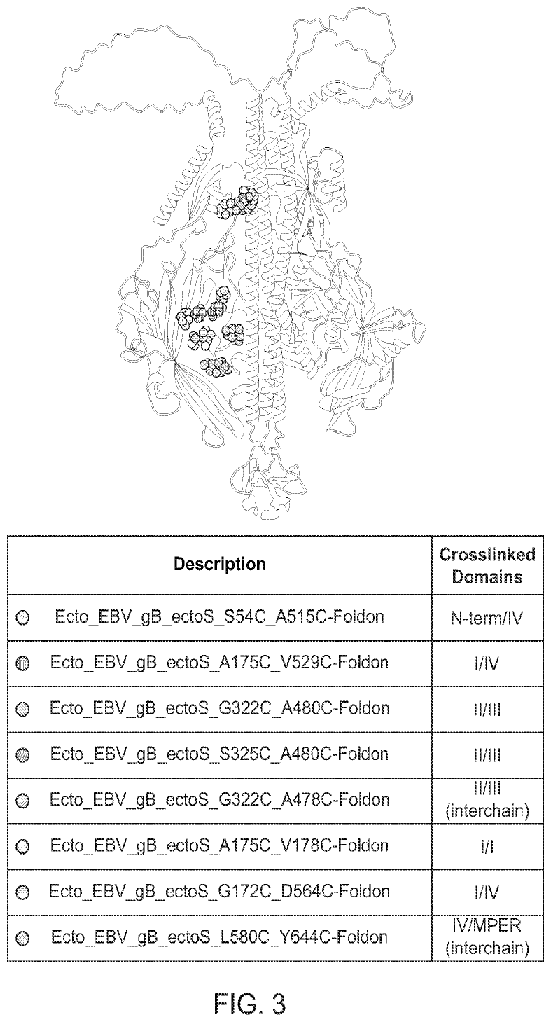

We generated a model of the metastable prefusion state of EBV gB with disulfide pairs identified in different colors and interdomain location descriptions summarized in FIG. 3.

We used a publicly available protein folding software program, Alphafold Multimer to generate a model of EBV gB in its prefusion state. We used the predicted model of EBV gB to help screen disulfides bonds that were predicted to form and calculated the root mean square deviation (RMSD) values in engineered regions to determine which disulfides would help lower the polypeptide's overall and local flexibility.

Using this methodology we were able to screen and prioritize predicted disulfide variants based off their ability to form disulfide bonds and their calculated RMSD values.

The result of producing a computation model and confirming formation of predicted disulfide bonds showed that our disulfide variants indeed formed, which helped in stabilizing a prefusion conformation of these EBV gB-derived polypeptides.

### Example 3

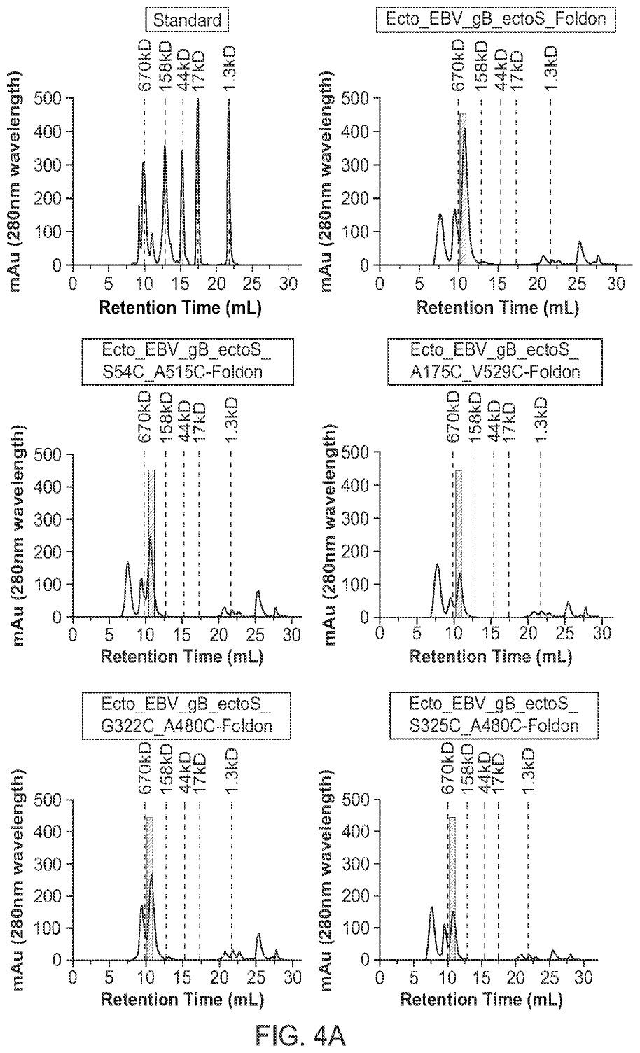

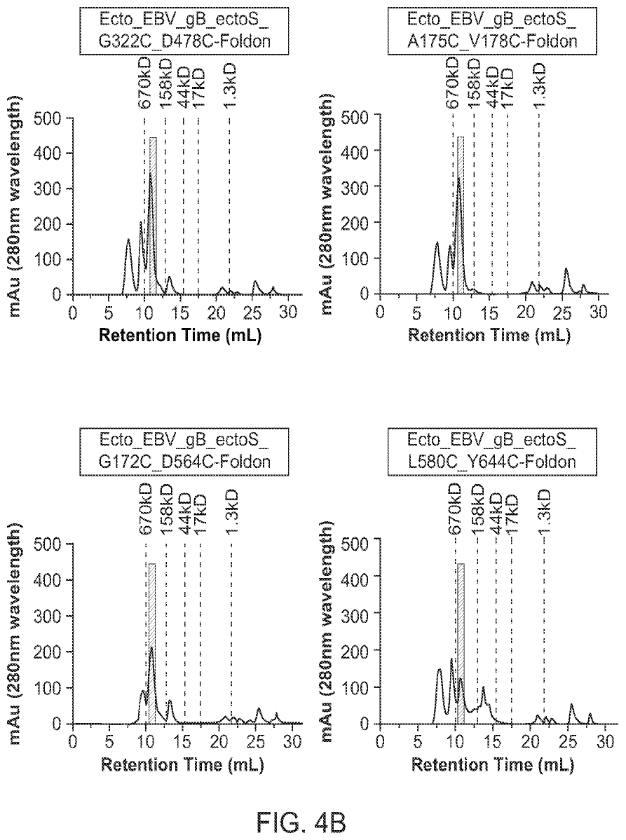

Disulfide-forming variants of EBV gB were produced, purified via Ni-NTA affinity chromatography (FIG. 4A, 4B), and characterized using size exclusion chromatography. We generated disulfide variants in HEK293F cells using the Daedalus mammalian expression system. The harvested supernatant from the expression culture was purified by Ni-NTA affinity chromatography and characterized via size exclusion chromatography.

Results illustrated a difference between the disulfide-forming variants of EBV gB in terms of the level of expression from crude Ni-purification, as well as the level of aggregate species. These results indicate our disulfide-forming variants were expressed and were capable of being purified using Ni-NTA affinity chromatography and size exclusion chromatography. Expression level was indicated by the intensity of Coomassie stain signal in the SDS PAGE images.

### Example 4

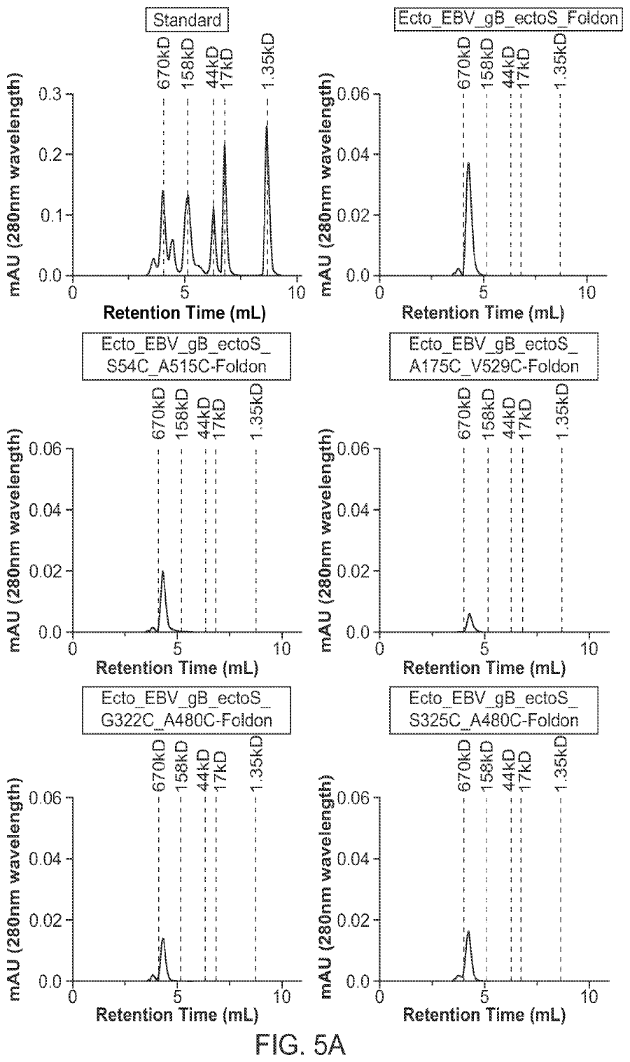

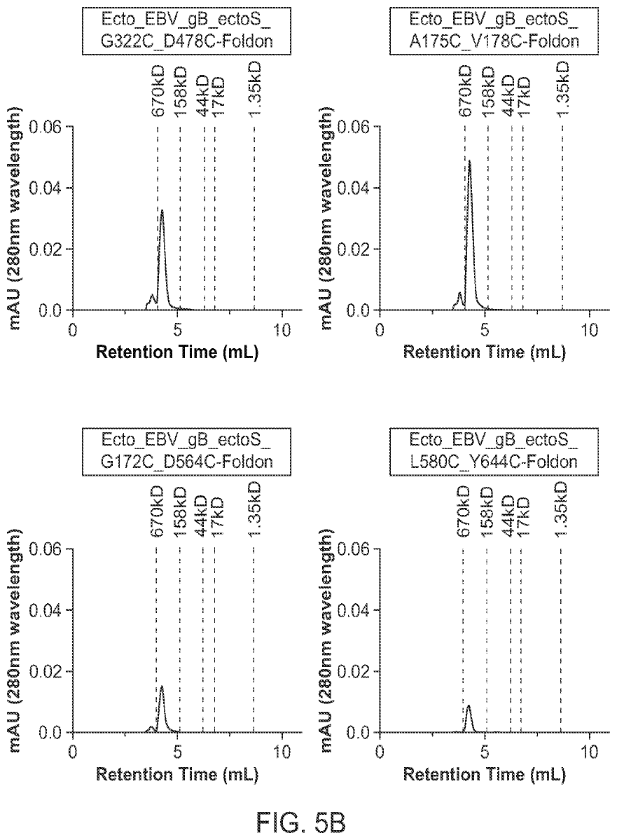

Disulfide-forming EBV gB variants were further purified using size exclusion chromatography to remove any aggregated or secondary species (FIG. 5A, 5B). Fractions which represented our disulfide-forming EBV gB variants were collected from the prior step of size exclusion chromatography run as shown in FIGS. 4A and 4B; and those fractions were further purified via size exclusion chromatography to produce the final material.

Clean peaks were generated to represent final product of disulfide-forming variants, which could then be further characterized via SDS-PAGE and negative stain electron microscopy. These results indicated successful purification of our disulfide-forming variants with clean peaks.

### Example 5. EBV gB Disulfide Stabilization Demonstrated by SDS-PAGE

We assayed the expression product after size exclusion chromatography purification to assess the formation of disulfide bond and characterization under reducing and nonreducing conditions.

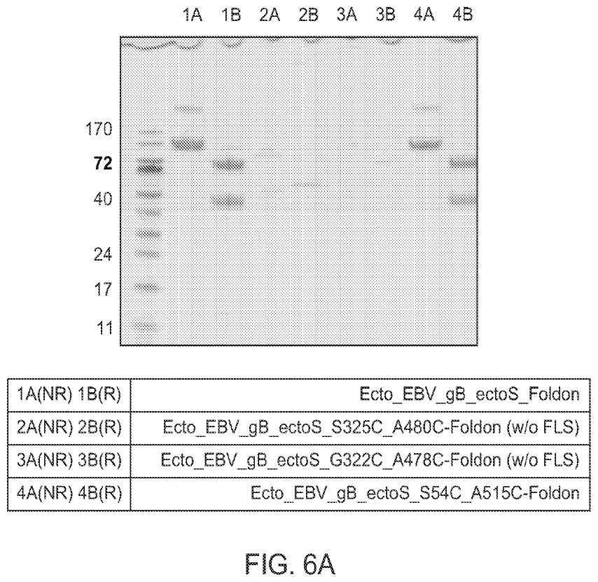

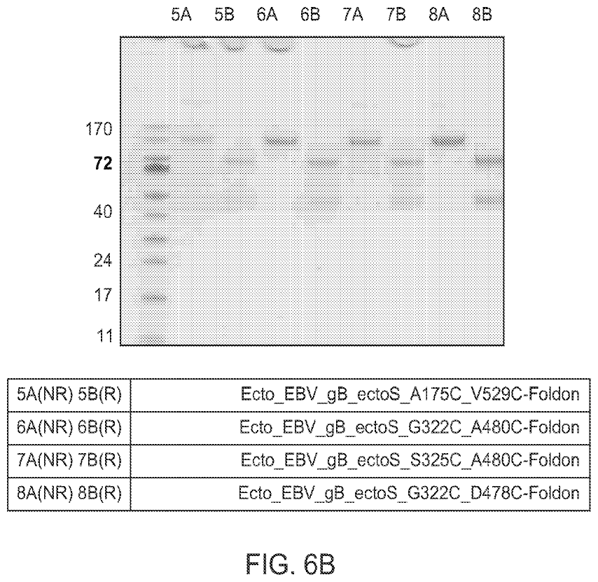

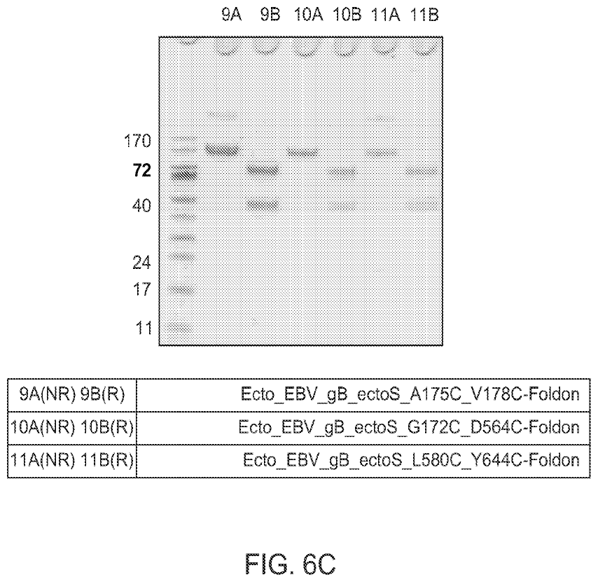

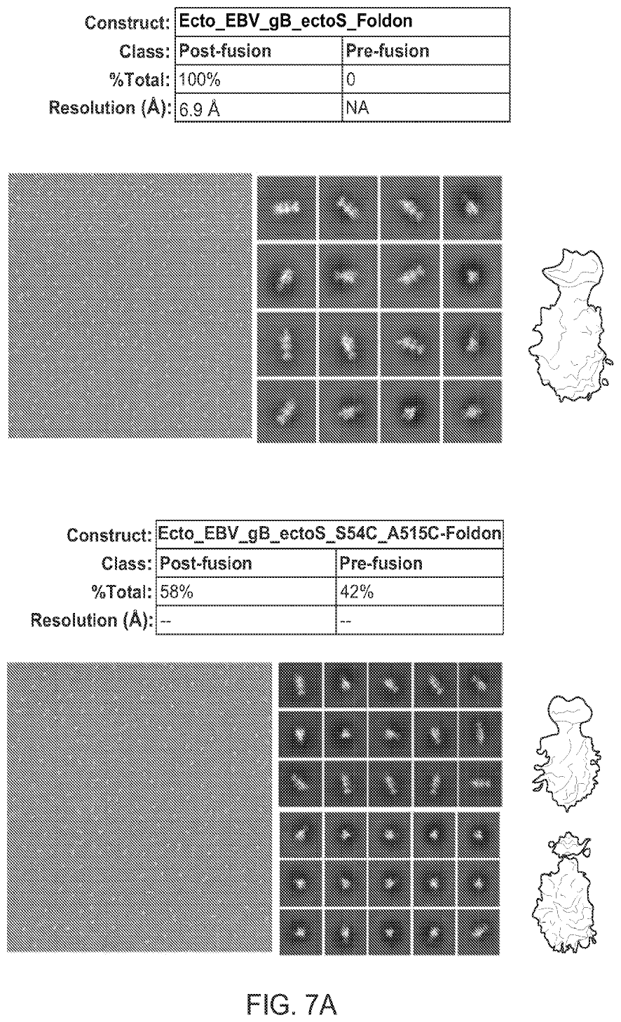

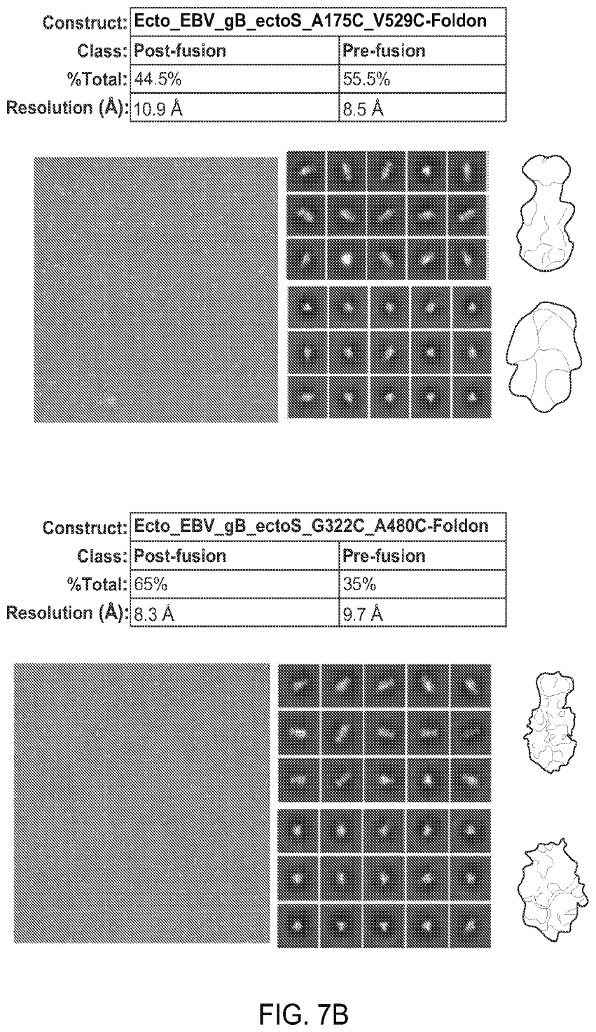

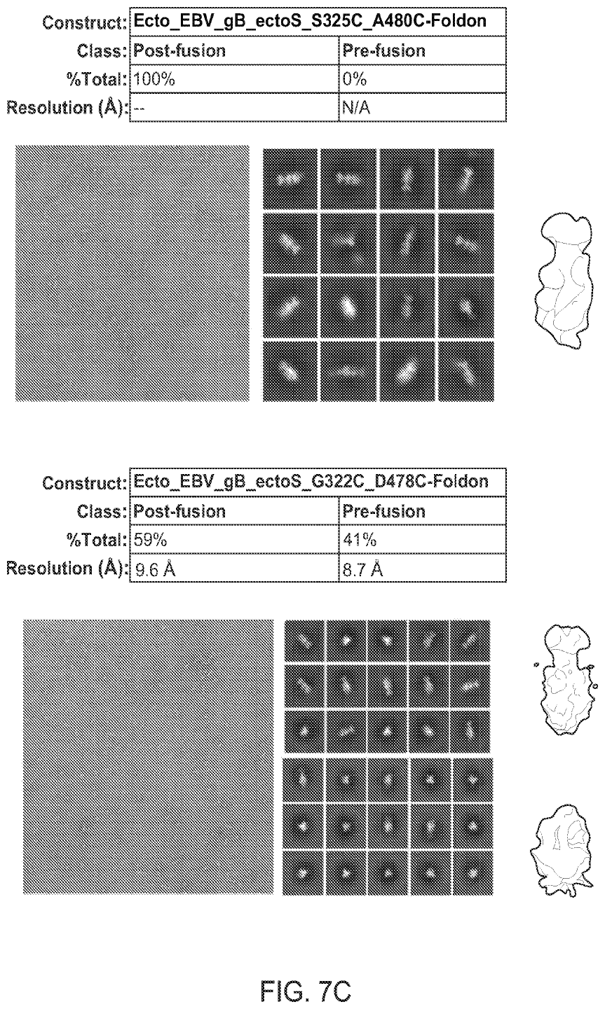

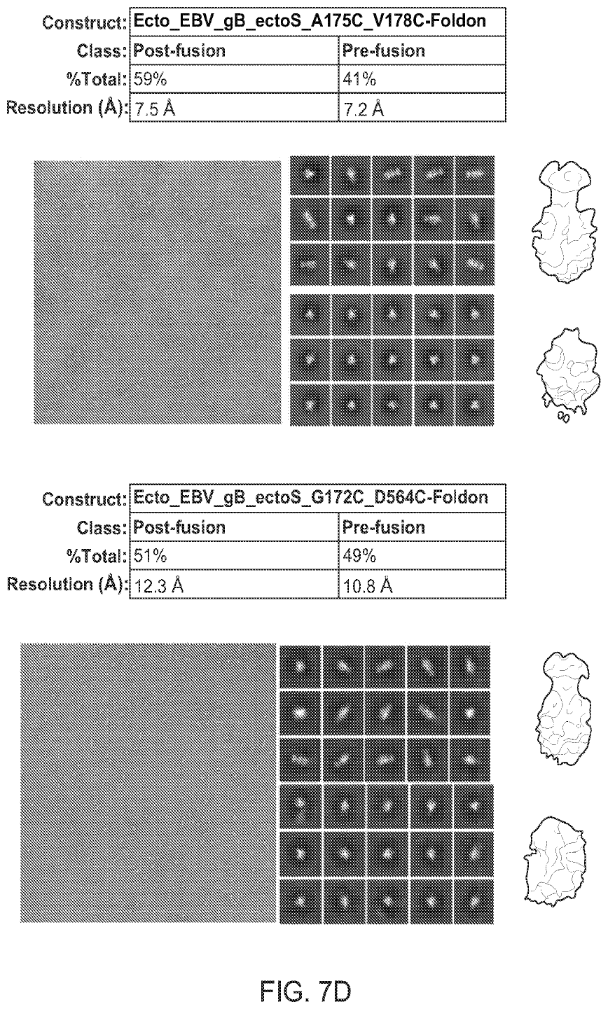

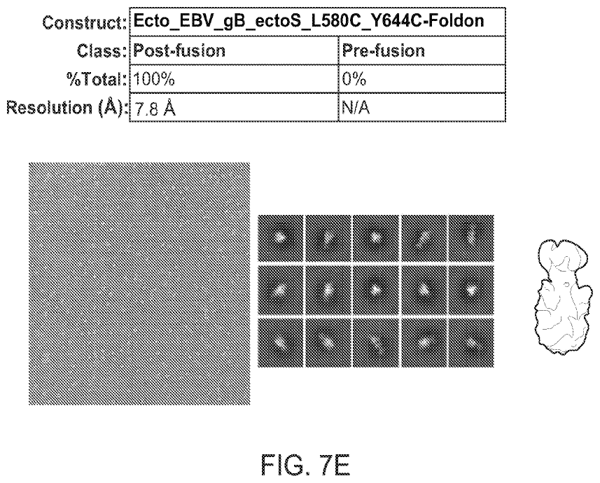

SDS-PAGE under reducing and non-reducing conditions was run with final SEC purified product (FIG. 6A-6C).

These results suggest our disulfide engineered constructs can express well and to varying degree, did not contain any contaminating bands, and formed disulfide bonds.

### Example 6. EBV gB Disulfide Stabilization-Negative Stain EM

Negative stain EM was performed with the purified product.

Negative stain analysis revealed that introduction of some of our engineered disulfides shifted the percent population that resembled the prefusion-like structure of EBV gB when compared to our control Ecto_EBV_gB_Foldon which contained no engineered disulfides. Of those analyzed by negative staining, the wild type, the one with amino acid substitutions S325C and A48C relative to native EBV gB, and the one with amino acid substitutions L580C and Y644C relative to native EBV gB, did not show prefusion stabilized structures in the EM imaging. This result showed that our engineered disulfide bonds stabilize a prefusion conformation of EBV gB variants.

### Example 7. Multiplex, Combinatorial Disulfide-Forming Variants of EBV gB

We designed prefusion conformation stabilized EBV gB variants resulting in a percent shift in comparison to wild-type in prefusion-like structures. From 0% prefusion-like structures to ˜50% when stabilizing disulfides were introduced. To further the percentage of the population that is in the prefusion-like state, we designed combinatorial disulfide variants. The constructs of disulfide-forming, stabilized EBV gB variants included a fibritin foldon trimerization domain and poly His affinity tags at the c-terminus. Two sets of constructs were designed with one set containing the native fusion loop amino acid sequences and a second set containing fusion loop substitutions (e.g., SEQ ID NOs:23-26).

The lead stabilized construct from single paired, disulfide-forming sequences were taken and combined to develop four new combinatorial disulfide stabilized constructs. Four combinatorial disulfide stabilized constructs were designed with fusion loop substitutions (e.g., SEQ ID NOs:23-26). This design strategy could shift the population of prefusion-like structure from 50% to 100% of the population, which further advanced suitability for development into protein vaccine as well as nucleic acid vaccines such as mRNA-based vaccines (with open reading frame encoding the disulfide stabilized polypeptides).

### Polypeptide Amino Acid Sequence Listings

1. Amino acid sequence of wildtype EBV gB subunit/monomer (Uniprot ID: P03188):

(SEQ ID NO: 1)
MTRRRVLSVVVLLAALACRLGAQTPEQPAPPATTVQPTATRQQTSFPFR
VCELSSHGDLFRFSSDIQCPSFGTRENHTEGLLMVFKDNIIPYSFKVRS
YTKIVTNILIYNGWYADSVTNRHEEKFSVDSYETDQMDTIYQCYNAVKM
TKDGLTRVYVDRDGVNITVNLKPTGGLANGVRRYASQTELYDAPGWLIW
TYRTRTTVNCLITDMMAKSNSPFDFFVTTTGQTVEMSPFYDGKNKETFH
ERADSFHVRTNYKIVDYDNRGTNPQGERRAFLDKGTYTLSWKLENRTAY
CPLQHWQTFDSTIATETGKSIHFVTDEGTSSFVTNTTVGIELPDAFKCI
EEQVNKTMHEKYEAVQDRYTKGQEAITYFITSGGLLLAWLPLTPRSLAT
VKNLTELTTPTSSPPSSPSPPAPSAARGSTPAAVLRRRRRDAGNATTPV
PPTAPGKSLGTLNNPATVQIQFAYDSLRRQINRMLGDLARAWCLEQKRQ
NMVLRELTKINPTTVMSSIYGKAVAAKRLGDVISVSQCVPVNQATVTLR
KSMRVPGSETMCYSRPLVSFSFINDTKTYEGQLGTDNEIFLTKKMTEVC
QATSQYYFQSGNEIHVYNDYHHFKTIELDGIATLQTFISLNTSLIENID
FASLELYSRDEQRASNVFDLEGIFREYNFQAQNIAGLRKDLDNAVSNGR
NQFVDGLGELMDSLGSVGQSITNLVSTVGGLFSSLVSGFISFFKNPFGG
MLILVLVAGVVILVISLTRRTRQMSQQPVQMLYPGIDELAQQHASGEGP
GINPISKTELQAIMLALHEQNQEQKRAAQRAAGPSVASRALQAARDRFP
GLRRRRYHDPETAAALLGEAETEF.

2. Amino acid sequence of an engineered polypeptide, having an altered EBV gB that has double mutation S54C/A515C (represented in bold and an increased font size) relative to the wildtype EBV strain GD1, wherein the amino acid numbering is based on amino acid positions in SEQ ID NO:1:

(SEQ ID NO: 2)
MTRRRVLSVVVLLAALACRLGAQTPEQPAPPATTVQPTATRQQTSFPFR
VCELCSHGDLFRFSSDIQCPSFGTRENHTEGLLMVFKDNIIPYSFKVRS
YTKIVTNILIYNGWYADSVTNRHEEKFSVDSYETDQMDTIYQCYNAVKM
TKDGLTRVYVDRDGVNITVNLKPTGGLANGVRRYASQTELYDAPGWLIW
TYRTRTTVNCLITDMMAKSNSPFDFFVTTTGQTVEMSPFYDGKNKETFH
ERADSFHVRTNYKIVDYDNRGTNPQGERRAFLDKGTYTLSWKLENRTAY
CPLQHWQTFDSTIATETGKSIHFVTDEGTSSFVTNTTVGIELPDAFKCI
EEQVNKTMHEKYEAVQDRYTKGQEAITYFITSGGLLLAWLPLTPRSLAT
VKNLTELTTPTSSPPSSPSPPAPSAARGSTPAAVLRRRRRDAGNATTPV
PPTAPGKSLGTLNNPATVQIQFAYDSLRRQINRMLGDLARAWCLEQKRQ
NMVLRELTKINPTTVMSSIYGKAVCAKRLGDVISVSQCVPVNQATVTLR
KSMRVPGSETMCYSRPLVSFSFINDTKTYEGQLGTDNEIFLTKKMTEVC
QATSQYYFQSGNEIHVYNDYHHFKTIELDGIATLQTFISLNTSLIENID
FASLELYSRDEQRASNVFDLEGIFREYNFQAQNIAGLRKDLDNAVSNGR
NQFVDGLGELMDSLGSVGQSITNLVSTVGGLFSSLVSGFISFFKNPFGG
MLILVLVAGVVILVISLTRRTRQMSQQPVQMLYPGIDELAQQHASGEGP
GINPISKTELQAIMLALHEQNQEQKRAAQRAAGPSVASRALQAARDRFP
GLRRRRYHDPETAAALLGEAETEF.

3. Amino acid sequence of an engineered polypeptide, having an altered EBV gB monomer sequence that has double mutation S55C/G227C (represented in bold and an increased font size) relative to the wildtype EBV strain GD1, wherein the amino acid numbering is based on amino acid positions in SEQ ID NO:1:

(SEQ ID NO: 3)
MTRRRVLSVVVLLAALACRLGAQTPEQPAPPATTVQPTATRQQTS
FPFRVCELS [symbol] HGDLFRFSSDIQCPSFGTRENHTEGLLMVFKDNII
PYSFKVRSYTKIVTNILIYNGWYADSVTNRHEEKFSVDSYETDQM
DTIYQCYNAVKMTKDGLTRVYVDRDGVNITVNLKPTGGLANGVRR
YASQTELYDAPGWLIWTYRTRTTVNCLITDMMAKSNSPFDFFVTT
T [symbol] QTVEMSPFYDGKNKETFHERADSFHVRTNYKIVDYDNRGTNPQ
GERRAFLDKGTYTLSWKLENRTAYCPLQHWQTFDSTIATETGKSI
HFVTDEGTSSFVTNTTVGIELPDAFKCIEEQVNKTMHEKYEAVQD
RYTKGQEAITYFITSGGLLLAWLPLTPRSLATVKNLTELTTPTSS
PPSSPSPPAPSAARGSTPAAVLRRRRRDAGNATTPVPPTAPGKSL
GTLNNPATVQIQFAYDSLRRQINRMLGDLARAWCLEQKRQNMVLR
ELTKINPTTVMSSIYGKAVAAKRLGDVISVSQCVPVNQATVTLRK
SMRVPGSETMCYSRPLVSFSFINDTKTYEGQLGTDNEIFLTKKMT
EVCQATSQYYFQSGNEIHVYNDYHHFKTIELDGIATLQTFISLNT
SLIENIDFASLELYSRDEQRASNVFDLEGIFREYNFQAQNIAGLR
KDLDNAVSNGRNQFVDGLGELMDSLGSVGQSITNLVSTVGGLFSS
LVSGFISFFKNPFGGMLILVLVAGVVILVISLTRRTRQMSQQPVQ
MLYPGIDELAQQHASGEGPGINPISKTELQAIMLALHEQNQEQKR
AAQRAAGPSVASRALQAARDRFPGLRRRRYHDPETAAALLGEAET
EF

4. Amino acid sequence of an engineered polypeptide, having an altered EBV gB sequence that has double mutation H56C/G227C (represented in bold and an increased font size) relative to the wildtype EBV strain GD1, wherein the amino acid numbering is based on amino acid positions in SEQ ID NO:1:

(SEQ ID NO: 4)
MTRRRVLSVVVLLAALACRLGAQTPEQPAPPATTVQPTATRQQTS
FPFRVCELSS [symbol] GDLFRFSSDIQCPSFGTRENHTEGLLMVFKDNII
PYSFKVRSYTKIVTNILIYNGWYADSVTNRHEEKFSVDSYETDQM
DTIYQCYNAVKMTKDGLTRVYVDRDGVNITVNLKPTGGLANGVRR
YASQTELYDAPGWLIWTYRTRTTVNCLITDMMAKSNSPFDFFVTT
T [symbol] QTVEMSPFYDGKNKETFHERADSFHVRTNYKIVDYDNRGTNPQ
GERRAFLDKGTYTLSWKLENRTAYCPLQHWQTFDSTIATETGKSI
HFVTDEGTSSFVTNTTVGIELPDAFKCIEEQVNKTMHEKYEAVQD
RYTKGQEAITYFITSGGLLLAWLPLTPRSLATVKNLTELTTPTSS
PPSSPSPPAPSAARGSTPAAVLRRRRRDAGNATTPVPPTAPGKSL
GTLNNPATVQIQFAYDSLRRQINRMLGDLARAWCLEQKRQNMVLR
ELTKINPTTVMSSIYGKAVAAKRLGDVISVSQCVPVNQATVTLRK
SMRVPGSETMCYSRPLVSFSFINDTKTYEGQLGTDNEIFLTKKMT
EVCQATSQYYFQSGNEIHVYNDYHHFKTIELDGIATLQTFISLNT
SLIENIDFASLELYSRDEQRASNVFDLEGIFREYNFQAQNIAGLR
KDLDNAVSNGRNQFVDGLGELMDSLGSVGQSITNLVSTVGGLFSS
LVSGFISFFKNPFGGMLILVLVAGVVILVISLTRRTRQMSQQPVQ
MLYPGIDELAQQHASGEGPGINPISKTELQAIMLALHEQNQEQKR
AAQRAAGPSVASRALQAARDRFPGLRRRRYHDPETAAALLGEAET
EF

5. Amino acid sequence of an engineered polypeptide, having an altered EBV gB monomer sequence that has double mutation A175C/V529C (represented in bold and an increased font size) relative to the wildtype EBV strain GD1, wherein the amino acid numbering is based on amino acid positions in SEQ ID NO:1:

(SEQ ID NO: 5)
MTRRRVLSVVVLLAALACRLGAQTPEQPAPPATTVQPTATRQQTS
FPFRVCELSSHGDLFRFSSDIQCPSFGTRENHTEGLLMVFKDNII
PYSFKVRSYTKIVTNILIYNGWYADSVTNRHEEKFSVDSYETDQM
DTIYQCYNAVKMTKDGLTRVYVDRDGVNITVNLKPTGGL [symbol] NGVRR
YASQTELYDAPGWLIWTYRTRTTVNCLITDMMAKSNSPFDFFVTT
TGQTVEMSPFYDGKNKETFHERADSFHVRTNYKIVDYDNRGTNPQ
GERRAFLDKGTYTLSWKLENRTAYCPLQHWQTFDSTIATETGKSI
HFVTDEGTSSFVTNTTVGIELPDAFKCIEEQVNKTMHEKYEAVQD
RYTKGQEAITYFITSGGLLLAWLPLTPRSLATVKNLTELTTPTSS
PPSSPSPPAPSAARGSTPAAVLRRRRRDAGNATTPVPPTAPGKSL
GTLNNPATVQIQFAYDSLRRQINRMLGDLARAWCLEQKRQNMVLR
ELTKINPTTVMSSIYGKAVAAKRLGDVISVSQC [symbol] PVNQATVTLRK
SMRVPGSETMCYSRPLVSFSFINDTKTYEGQLGTDNEIFLTKKMT
EVCQATSQYYFQSGNEIHVYNDYHHFKTIELDGIATLQTFISLNT
SLIENIDFASLELYSRDEQRASNVFDLEGIFREYNFQAQNIAGLR
KDLDNAVSNGRNQFVDGLGELMDSLGSVGQSITNLVSTVGGLFSS
LVSGFISFFKNPFGGMLILVLVAGVVILVISLTRRTRQMSQQPVQ
MLYPGIDELAQQHASGEGPGINPISKTELQAIMLALHEQNQEQKR
AAQRAAGPSVASRALQAARDRFPGLRRRRYHDPETAAALLGEAET
EF.

6. Amino acid sequence of an engineered polypeptide, having an altered EBV gB sequence that has double mutation G322C/A480C (represented in bold and an increased font size) relative to the wildtype EBV strain GD1, wherein the amino acid numbering is based on amino acid positions in SEQ ID NO:1:

(SEQ ID NO: 6)
MTRRRVLSVVVLLAALACRLGAQTPEQPAPPATTVQPTATRQQTS
FPFRVCELSSHGDLFRFSSDIQCPSFGTRENHTEGLLMVFKDNII
PYSFKVRSYTKIVTNILIYNGWYADSVTNRHEEKFSVDSYETDQM
DTIYQCYNAVKMTKDGLTRVYVDRDGVNITVNLKPTGGLANGVRR
YASQTELYDAPGWLIWTYRTRTTVNCLITDMMAKSNSPFDFFVTT
TGQTVEMSPFYDGKNKETFHERADSFHVRTNYKIVDYDNRGTNPQ
GERRAFLDKGTYTLSWKLENRTAYCPLQHWQTFDSTIATETGKSI
HFVTDE [symbol] TSSFVTNTTVGIELPDAFKCIEEQVNKTMHEKYEAVQD
RYTKGQEAITYFITSGGLLLAWLPLTPRSLATVKNLTELTTPTSS
PPSSPSPPAPSAARGSTPAAVLRRRRRDAGNATTPVPPTAPGKSL
GTLNNPATVQIQFAYDSLRRQINRMLGDL [symbol] RAWCLEQKRQNMVLR
ELTKINPTTVMSSIYGKAVAAKRLGDVISVSQCVPVNQATVTLRK
SMRVPGSETMCYSRPLVSFSFINDTKTYEGQLGTDNEIFLTKKMT
EVCQATSQYYFQSGNEIHVYNDYHHFKTIELDGIATLQTFISLNT
SLIENIDFASLELYSRDEQRASNVFDLEGIFREYNFQAQNIAGLR
KDLDNAVSNGRNQFVDGLGELMDSLGSVGQSITNLVSTVGGLFSS
LVSGFISFFKNPFGGMLILVLVAGVVILVISLTRRTRQMSQQPVQ
MLYPGIDELAQQHASGEGPGINPISKTELQAIMLALHEQNQEQKR
AAQRAAGPSVASRALQAARDRFPGLRRRRYHDPETAAALLGEAET
EF

7. Amino acid sequence of an engineered polypeptide, having an altered EBV gB sequence that has double mutation S325C/A480C (represented in bold and an increased font size) relative to the wildtype EBV strain GD1, wherein the amino acid numbering is based on amino acid positions in SEQ ID NO:1:

(SEQ ID NO: 7)
MTRRRVLSVVVLLAALACRLGAQTPEQPAPPATTVQPTATRQQTS
FPFRVCELSSHGDLFRFSSDIQCPSFGTRENHTEGLLMVFKDNII
PYSFKVRSYTKIVTNILIYNGWYADSVTNRHEEKFSVDSYETDQM
DTIYQCYNAVKMTKDGLTRVYVDRDGVNITVNLKPTGGLANGVRR
YASQTELYDAPGWLIWTYRTRTTVNCLITDMMAKSNSPFDFFVTT
TGQTVEMSPFYDGKNKETFHERADSFHVRTNYKIVDYDNRGTNPQ
GERRAFLDKGTYTLSWKLENRTAYCPLQHWQTFDSTIATETGKSI
HFVTDEGTS [symbol] FVTNTTVGIELPDAFKCIEEQVNKTMHEKYEAVQD
RYTKGQEAITYFITSGGLLLAWLPLTPRSLATVKNLTELTTPTSS
PPSSPSPPAPSAARGSTPAAVLRRRRRDAGNATTPVPPTAPGKSL
GTLNNPATVQIQFAYDSLRRQINRMLGDL [symbol] RAWCLEQKRQNMVLR
ELTKINPTTVMSSIYGKAVAAKRLGDVISVSQCVPVNQATVTLRK
SMRVPGSETMCYSRPLVSFSFINDTKTYEGQLGTDNEIFLTKKMT
EVCQATSQYYFQSGNEIHVYNDYHHFKTIELDGIATLQTFISLNT
SLIENIDFASLELYSRDEQRASNVFDLEGIFREYNFQAQNIAGLR
KDLDNAVSNGRNQFVDGLGELMDSLGSVGQSITNLVSTVGGLFSS
LVSGFISFFKNPFGGMLILVLVAGVVILVISLTRRTRQMSQQPVQ
MLYPGIDELAQQHASGEGPGINPISKTELQAIMLALHEQNQEQKR
AAQRAAGPSVASRALQAARDRFPGLRRRRYHDPETAAALLGEAET
EF.

8. Amino acid sequence of an engineered polypeptide, having an altered EBV gB sequence that has double mutation G322C/D478C represented in bold and an increased font size) relative to the wildtype EBV strain GD1, wherein the amino acid numbering is based on amino acid positions in SEQ ID NO:1:

(SEQ ID NO: 8)
MTRRRVLSVVVLLAALACRLGAQTPEQPAPPATTVQPTATRQQTS
FPFRVCELSSHGDLFRFSSDIQCPSFGTRENHTEGLLMVFKDNII
PYSFKVRSYTKIVTNILIYNGWYADSVTNRHEEKFSVDSYETDQM
DTIYQCYNAVKMTKDGLTRVYVDRDGVNITVNLKPTGGLANGVRR
YASQTELYDAPGWLIWTYRTRTTVNCLITDMMAKSNSPFDFFVTT
TGQTVEMSPFYDGKNKETFHERADSFHVRTNYKIVDYDNRGTNPQ
GERRAFLDKGTYTLSWKLENRTAYCPLQHWQTFDSTIATETGKSI
HFVTDE [symbol] TSSFVTNTTVGIELPDAFKCIEEQVNKTMHEKYEAVQD
RYTKGQEAITYFITSGGLLLAWLPLTPRSLATVKNLTELTTPTSS
PPSSPSPPAPSAARGSTPAAVLRRRRRDAGNATTPVPPTAPGKSL
GTLNNPATVQIQFAYDSLRRQINRMLG [symbol] LARAWCLEQKRQNMVLR
ELTKINPTTVMSSIYGKAVAAKRLGDVISVSQCVPVNQATVTLRK
SMRVPGSETMCYSRPLVSFSFINDTKTYEGQLGTDNEIFLTKKMT
EVCQATSQYYFQSGNEIHVYNDYHHFKTIELDGIATLQTFISLNT
SLIENIDFASLELYSRDEQRASNVFDLEGIFREYNFQAQNIAGLR
KDLDNAVSNGRNQFVDGLGELMDSLGSVGQSITNLVSTVGGLFSS
LVSGFISFFKNPFGGMLILVLVAGVVILVISLTRRTRQMSQQPVQ
MLYPGIDELAQQHASGEGPGINPISKTELQAIMLALHEQNQEQKR
AAQRAAGPSVASRALQAARDRFPGLRRRRYHDPETAAALLGEAET
EF.

9. Amino acid sequence of an engineered polypeptide, having an altered EBV gB sequence that has double mutation L696C/S727C (represented in bold and an increased font size) relative to the wildtype EBV strain GD1, wherein the amino acid numbering is based on amino acid positions in SEQ ID NO:1:

(SEQ ID NO: 9)
MTRRRVLSVVVLLAALACRLGAQTPEQPAPPATTVQPTATRQQTS
FPFRVCELSSHGDLFRFSSDIQCPSFGTRENHTEGLLMVFKDNII
PYSFKVRSYTKIVTNILIYNGWYADSVTNRHEEKFSVDSYETDQM
DTIYQCYNAVKMTKDGLTRVYVDRDGVNITVNLKPTGGLANGVRR
YASQTELYDAPGWLIWTYRTRTTVNCLITDMMAKSNSPFDFFVTT
TGQTVEMSPFYDGKNKETFHERADSFHVRTNYKIVDYDNRGTNPQ
GERRAFLDKGTYTLSWKLENRTAYCPLQHWQTFDSTIATETGKSI
HFVTDEGTSSFVTNTTVGIELPDAFKCIEEQVNKTMHEKYEAVQD
RYTKGQEAITYFITSGGLLLAWLPLTPRSLATVKNLTELTTPTSS
PPSSPSPPAPSAARGSTPAAVLRRRRRDAGNATTPVPPTAPGKSL
GTLNNPATVQIQFAYDSLRRQINRMLGDLARAWCLEQKRQNMVLR
ELTKINPTTVMSSIYGKAVAAKRLGDVISVSQCVPVNQATVTLRK
SMRVPGSETMCYSRPLVSFSFINDTKTYEGQLGTDNEIFLTKKMT
EVCQATSQYYFQSGNEIHVYNDYHHFKTIELDGIATLQTFISLNT
SLIENIDFASLELYSRDEQRASNVFDLEGIFREYNFQAQNIAGLR
KDLDNAVSNGRNQFVDGLGE [symbol] MDSLGSVGQSITNLVSTVGGLFSS
LVSGFI [symbol] FFKNPFGGMLILVLVAGVVILVISLTRRTRQMSQQPVQ
MLYPGIDELAQQHASGEGPGINPISKTELQAIMLALHEQNQEQKR
AAQRAAGPSVASRALQAARDRFPGLRRRRYHDPETAAALLGEAET
EF.

10. Amino acid sequence of an engineered polypeptide, having an altered EBV gB sequence that has double mutation A175C/V178C (represented in bold and an increased font size) relative to the wildtype EBV strain GD1, wherein the amino acid numbering is based on amino acid positions in SEQ ID NO:1:

(SEQ ID NO: 10)
MTRRRVLSVVVLLAALACRLGAQTPEQPAPPATTVQPTATRQQTS
FPFRVCELSSHGDLFRFSSDIQCPSFGTRENHTEGLLMVFKDNII
PYSFKVRSYTKIVTNILIYNGWYADSVTNRHEEKFSVDSYETDQM
DTIYQCYNAVKMTKDGLTRVYVDRDGVNITVNLKPTGGL [symbol] NG [symbol] RR
YASQTELYDAPGWLIWTYRTRTTVNCLITDMMAKSNSPFDFFVTT
TGQTVEMSPFYDGKNKETFHERADSFHVRTNYKIVDYDNRGTNPQ
GERRAFLDKGTYTLSWKLENRTAYCPLQHWQTFDSTIATETGKSI
HFVTDEGTSSFVTNTTVGIELPDAFKCIEEQVNKTMHEKYEAVQD
RYTKGQEAITYFITSGGLLLAWLPLTPRSLATVKNLTELTTPTSS
PPSSPSPPAPSAARGSTPAAVLRRRRRDAGNATTPVPPTAPGKSL
GTLNNPATVQIQFAYDSLRRQINRMLGDLARAWCLEQKRQNMVLR
ELTKINPTTVMSSIYGKAVAAKRLGDVISVSQCVPVNQATVTLRK
SMRVPGSETMCYSRPLVSFSFINDTKTYEGQLGTDNEIFLTKKMT
EVCQATSQYYFQSGNEIHVYNDYHHFKTIELDGIATLQTFISLNT
SLIENIDFASLELYSRDEQRASNVFDLEGIFREYNFQAQNIAGLR
KDLDNAVSNGRNQFVDGLGELMDSLGSVGQSITNLVSTVGGLFSS
LVSGFISFFKNPFGGMLILVLVAGVVILVISLTRRTRQMSQQPVQ
MLYPGIDELAQQHASGEGPGINPISKTELQAIMLALHEQNQEQKR
AAQRAAGPSVASRALQAARDRFPGLRRRRYHDPETAAALLGEAET
EF.

11. Amino acid sequence of an engineered polypeptide, having an altered EBV gB sequence that has double mutation G172C/D564C (represented in bold and an increased font size) relative to the wildtype EBV strain GD1, wherein the amino acid numbering is based on amino acid positions in SEQ ID NO:1:

(SEQ ID NO: 11)
MTRRRVLSVVVLLAALACRLGAQTPEQPAPPATTVQPTATRQQTS
FPFRVCELSSHGDLFRFSSDIQCPSFGTRENHTEGLLMVFKDNII
PYSFKVRSYTKIVTNILIYNGWYADSVTNRHEEKFSVDSYETDQM
DTIYQCYNAVKMTKDGLTRVYVDRDGVNITVNLKPT [symbol] GLANGVRR
YASQTELYDAPGWLIWTYRTRTTVNCLITDMMAKSNSPFDFFVTT
TGQTVEMSPFYDGKNKETFHERADSFHVRTNYKIVDYDNRGTNPQ
GERRAFLDKGTYTLSWKLENRTAYCPLQHWQTFDSTIATETGKSI
HFVTDEGTSSFVTNTTVGIELPDAFKCIEEQVNKTMHEKYEAVQD
RYTKGQEAITYFITSGGLLLAWLPLTPRSLATVKNLTELTTPTSS
PPSSPSPPAPSAARGSTPAAVLRRRRRDAGNATTPVPPTAPGKSL
GTLNNPATVQIQFAYDSLRRQINRMLGDLARAWCLEQKRQNMVLR
ELTKINPTTVMSSIYGKAVAAKRLGDVISVSQCVPVNQATVTLRK
SMRVPGSETMCYSRPLVSFSFIN [symbol] TKTYEGQLGTDNEIFLTKKMT
EVCQATSQYYFQSGNEIHVYNDYHHFKTIELDGIATLQTFISLNT
SLIENIDFASLELYSRDEQRASNVFDLEGIFREYNFQAQNIAGLR
KDLDNAVSNGRNQFVDGLGELMDSLGSVGQSITNLVSTVGGLFSS
LVSGFISFFKNPFGGMLILVLVAGVVILVISLTRRTRQMSQQPVQ
MLYPGIDELAQQHASGEGPGINPISKTELQAIMLALHEQNQEQKR
AAQRAAGPSVASRALQAARDRFPGLRRRRYHDPETAAALLGEAET
EF.

12. Amino acid sequence of an engineered polypeptide, having an altered EBV gB sequence that has double mutation L580C/Y644C (represented in bold and an increased font size) relative to the wildtype EBV strain GD1, wherein the amino acid numbering is based on amino acid positions in SEQ ID NO:1:

(SEQ ID NO: 12)
MTRRRVLSVVVLLAALACRLGAQTPEQPAPPATTVQPTATRQQTS
FPFRVCELSSHGDLFRFSSDIQCPSFGTRENHTEGLLMVFKDNII
PYSFKVRSYTKIVTNILIYNGWYADSVTNRHEEKFSVDSYETDQM
DTIYQCYNAVKMTKDGLTRVYVDRDGVNITVNLKPTGGLANGVRR
YASQTELYDAPGWLIWTYRTRTTVNCLITDMMAKSNSPFDFFVTT
TGQTVEMSPFYDGKNKETFHERADSFHVRTNYKIVDYDNRGTNPQ
GERRAFLDKGTYTLSWKLENRTAYCPLQHWQTFDSTIATETGKSI
HFVTDEGTSSFVTNTTVGIELPDAFKCIEEQVNKTMHEKYEAVQD
RYTKGQEAITYFITSGGLLLAWLPLTPRSLATVKNLTELTTPTSS
PPSSPSPPAPSAARGSTPAAVLRRRRRDAGNATTPVPPTAPGKSL
GTLNNPATVQIQFAYDSLRRQINRMLGDLARAWCLEQKRQNMVLR
ELTKINPTTVMSSIYGKAVAAKRLGDVISVSQCVPVNQATVTLRK
SMRVPGSETMCYSRPLVSFSFINDTKTYEGQLGTDNEIF [symbol] TKKMT
EVCQATSQYYFQSGNEIHVYNDYHHFKTIELDGIATLQTFISLNT
SLIENIDFASLEL [symbol] SRDEQRASNVFDLEGIFREYNFQAQNIAGLR
KDLDNAVSNGRNQFVDGLGELMDSLGSVGQSITNLVSTVGGLFSS
LVSGFISFFKNPFGGMLILVLVAGVVILVISLTRRTRQMSQQPVQ
MLYPGIDELAQQHASGEGPGINPISKTELQAIMLALHEQNQEQKR
AAQRAAGPSVASRALQAARDRFPGLRRRRYHDPETAAALLGEAET
EF.

13. Amino acid sequence of a polypeptide coined “Ecto_EBV_gB_ectoS_Foldon”, comprising an altered short ectodomain (EctoS) sequence that has fusion loop substitutions wherein the wild type residues WY¹¹²⁻¹¹³ and WLIW¹⁹³⁻¹⁹⁶ (SEQ ID NO:28) in SEQ ID NO: 1 are replaced with HR and RVEA (SEQ ID NO:29) shown in bolded italics below, wherein native EctoS sequences is positions 23-683 in SEQ ID NO:1; and this amino acid sequence is:

(SEQ ID NO:13)

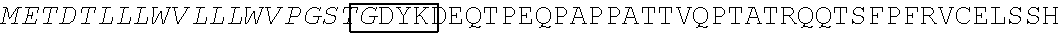

GDLFRFSSDIQCPSFGTRENHTEGLLMVFKDNIIPYSFKVRSYTKIVTNILIYNGHRAD
SVTNRHEEKFSVDSYETDQMDTIYQCYNAVKMTKDGLTRVYVDRDGVNITVNLKPT
GGLANGVRRYASQTELYDAPGRVEATYRTRTTVNCLITDMMAKSNSPFDFFVTTTGQ
TVEMSPFYDGKNKETFHERADSFHVRTNYKIVDYDNRGTNPQGERRAFLDKGTYTL
SWKLENRTAYCPLQHWQTFDSTIATETGKSIHFVTDEGTSSFVTNTTVGIELPDAFKCI
EEQVNKTMHEKYEAVQDRYTKGQEAITYFITSGGLLLAWLPLTPRSLATVKNLTELTT
PTSSPPSSPSPPAPPAARGSTSAAVLRRRRRDVGNATTPVPPAAPGKSLGTLNNPATVQI
QFAYDSLRRQINRMLGDLARAWCLEQKRQNMVLRELTKINPTTVMSSIYGKAVAAK
RLGDVISVSQCVPVNQATVTLRKSMRVPGSETMCYSRPLVSFSFINDTKTYEGQLGTD
NEIFLTKKMTEVCQATSQYYFQSGNEIHVYNDYHHFKTIELDGIATLQTFISLNTSLIEN

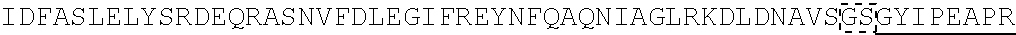

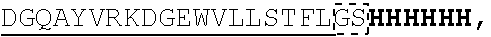

which, relative to the native EctoS sequence, further contains:
at the N-terminus, an Ig Kappa signal peptide sequence (the cleavable portion) in positions 1-20 herein (in non-bolded italics) and a short polypeptide marker sequence in positions 21-25 herein (boxed in solid line), and
at the C-terminus, a fibritin foldon trimerization domain in positions 689-715 herein (underlined, also individually listed as SEQ ID NO:32), a glycine-serine (GS) flexible linker (boxed in dashed line) flanking the fibritin foldon trimerization domain, and a poly histidine affinity tag in positions 718-723 herein (in bold, non-italicized and non-enlarged font size).
14. Amino acid sequence of a polypeptide coined “Ecto_EBV_gB_ectoS_S54C_A515C-Foldon”, comprising an altered EctoS sequence that has the fusion loop substitutions (shown in bolded italics) and two engineered sulfhydryl-containing amino acid residues (shown in bold and an increased font size), relative to the native EctoS sequence, wherein the two engineered sulfhydryl-containing amino acid residues are in positions 57 and 518 herein and are substitutions of cysteine amino acid residues for wild type residues S54 and A515 in SEQ ID NO: 1; and this amino acid sequence is:

(SEQ ID NO:14)

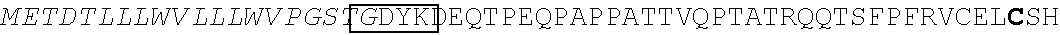

GDLFRFSSDIQCPSFGTRENHTEGLLMVFKDNIIPYSFKVRSYTKIVTNILIYNGHRAD
SVTNRHEEKFSVDSYETDQMDTIYQCYNAVKMTKDGLTRVYVDRDGVNITVNLKPT
GGLCNGVRRYASQTELYDAPGRVEATYRTRTTVNCLITDMMAKSNSPFDFFVTTTGQ
TVEMSPFYDGKNKETFHERADSFHVRTNYKIVDYDNRGTNPQGERRAFLDKGTYTL
SWKLENRTAYCPLQHWQTFDSTIATETGKSIHFVTDEGTSSFVTNTTVGIELPDAFKCI
EEQVNKTMHEKYEAVQDRYTKGQEAITYFITSGGLLLAWLPLTPRSLATVKNLTELTT
PTSSPPSSPSPPAPPAARGSTSAAVLRRRRRDVGNATTPVPPAAPGKSLGTLNNPATVQI
QFAYDSLRRQINRMLGDLARAWCLEQKRQNMVLRELTKINPTTVMSSIYGKAVCAK
RLGDVISVSQCVPVNQATVTLRKSMRVPGSETMCYSRPLVSFSFINDTKTYEGQLGTD
NEIFLTKKMTEVCQATSQYYFQSGNEIHVYNDYHHFKTIELDGIATLQTFISLNTSLIEN

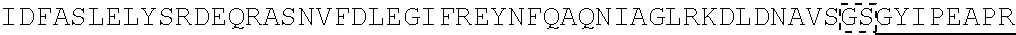

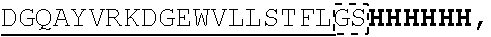

which, relative to the native EctoS sequence, further contains:
at the N-terminus, an Ig Kappa signal peptide sequence in positions 1-20 herein (in non-bolded italics) and a short polypeptide marker sequence in positions 21-25 herein (boxed in solid line), and at the C-terminus, a fibritin foldon trimerization domain in positions 689-715 herein (underlined), a glycine-serine (GS) flexible linker (boxed in dash line) flanking the fibritin foldon trimerization domain, and a poly histidine affinity tag in positions 718-723 herein (in bold, non-italicized and non-enlarged font size).
15. Amino acid sequence of a polypeptide coined “Ecto_EBV_gB_ectoS_A175C_V529C-Foldon”, comprising an altered EctoS sequence that has the fusion loop substitutions (shown in bolded italics) and two engineered sulfhydryl-containing amino acid residues (shown in bold and an increased font size), relative to the native EctoS sequence, wherein the two engineered sulfhydryl-containing amino acid residues are in positions 178 and 532 herein and are substitutions of cysteine amino acid residues for wild type residues A175 and V529 in SEQ ID NO:1; and this amino acid sequence is:

(SEQ ID NO:15)

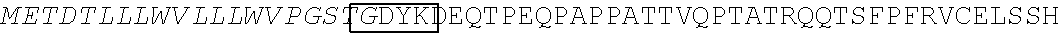

GDLFRFSSDIQCPSFGTRENHTEGLLMVFKDNIIPYSFKVRSYTKIVTNILIYNGHRAD
SVTNRHEEKFSVDSYETDQMDTIYQCYNAVKMTKDGLTRVYVDRDGVNITVNLKPT
GGLCNGVRRYASQTELYDAPGRVEATYRTRTTVNCLITDMMAKSNSPFDFFVTTTGQ
TVEMSPFYDGKNKETFHERADSFHVRTNYKIVDYDNRGTNPQGERRAFLDKGTYTL
SWKLENRTAYCPLQHWQTFDSTIATETGKSIHFVTDEGTSSFVTNTTVGIELPDAFKCI
EEQVNKTMHEKYEAVQDRYTKGQEAITYFITSGGLLLAWLPLTPRSLATVKNLTELTT
PTSSPPSSPSPPAPPAARGSTSAAVLRRRRRDVGNATTPVPPAAPGKSLGTLNNPATVQI
QFAYDSLRRQINRMLGDLARAWCLEQKRQNMVLRELTKINPTTVMSSIYGKAVAAK
RLGDVISVSQCCPVNQATVTLRKSMRVPGSETMCYSRPLVSFSFINDTKTYEGQLGT
DNEIFLTKKMTEVCQATSQYYFQSGNEIHVYNDYHHFKTIELDGIATLQTFISLNTSLIE

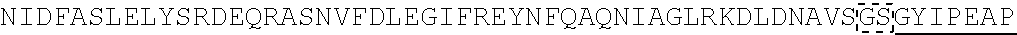

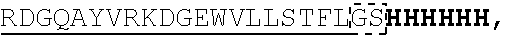

which, relative to the native EctoS sequence, further contains:
at the N-terminus, an Ig Kappa signal peptide sequence in positions 1-20 herein (in non-bolded italics) and a short polypeptide marker sequence in positions 21-25 herein (boxed in solid line), and
at the C-terminus, a fibritin foldon trimerization domain in positions 689-715 herein (underlined), a glycine-serine (GS) flexible linker (boxed in dashed line) flanking the fibritin foldon trimerization domain, and a poly histidine affinity tag in positions 718-723 herein (in bold, non-italicized and non-enlarged font size).
16. Amino acid sequence of a polypeptide coined “Ecto_EBV_gB_ectoS_G322C_A480C-Foldon”, comprising an altered EctoS sequence that has the fusion loop substitutions (shown in bolded italics) and two engineered sulfhydryl-containing amino acid residues (shown in bold and an increased font size), relative to the native EctoS sequence, wherein the two engineered sulfhydryl-containing amino acid residues are in positions 325 and 483 herein and are substitutions of cysteine amino acid residues for wild type residues G322 and A480 in SEQ ID NO:1; and this amino acid sequence is:

(SEQ ID NO: 16)

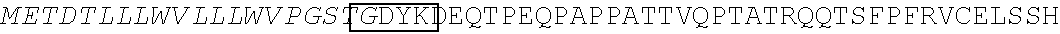

GDLFRFSSDIQCPSFGTRENHTEGLLMVFKDNIIPYSFKVRSYTKIVTNILIYNGHRAD
SVTNRHEEKFSVDSYETDQMDTIYQCYNAVKMTKDGLTRVYVDRDGVNITVNLKPT
GGLANGVRRYASQTELYDAPGRVEATYRTRTTVNCLITDMMAKSNSPFDFFVTTTGQ
TVEMSPFYDGKNKETFHERADSFHVRTNYKIVDYDNRGTNPQGERRAFLDKGTYTL
SWKLENRTAYCPLQHWQTFDSTIATETGKSIHFVTDECTSSFVTNTTVGIELPDAFKCI
EEQVNKTMHEKYEAVQDRYTKGQEAITYFITSGGLLLAWLPLTPRSLATVKNLTELTT
PTSSPPSSPSPPAPPAARGSTSAAVLRRRRRDVGNATTPVPPAAPGKSLGTLNNPATVQI
QFAYDSLRRQINRMLGDLCRAWCLEQKRQNMVLRELTKINPTTVMSSIYGKAVAAK
RLGDVISVSQCVPVNQATVTLRKSMRVPGSETMCYSRPLVSFSFINDTKTYEGQLGTD
NEIFLTKKMTEVCQATSQYYFQSGNEIHVYNDYHHFKTIELDGIATLQTFISLNTSLIEN

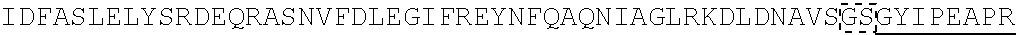

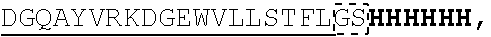

which, relative to the native EctoS sequence, further contains:
at the N-terminus, an Ig Kappa signal peptide sequence in positions 1-20 herein (in non-bolded italics) and a short polypeptide marker sequence in positions 21-25 herein (boxed in solid line), and
at the C-terminus, a fibritin foldon trimerization domain in positions 689-715 herein (underlined), a glycine-serine (GS) flexible linker (boxed in dashed line) flanking the fibritin foldon trimerization domain, and a poly histidine affinity tag in positions 718-723 herein (in bold, non-italicized and non-enlarged font size).
17. Amino acid sequence of a polypeptide coined “Ecto_EBV_gB_ectoS_S325C_A480C-Foldon”, comprising an altered EctoS sequence that has the fusion loop substitutions (shown in bolded italics) and two engineered sulfhydryl-containing amino acid residues (shown in bold and an increased font size), relative to the native EctoS sequence, wherein the two engineered sulfhydryl-containing amino acid residues are in positions 328 and 483 herein and are substitutions of cysteine amino acid residues for wild type residues S325 and A480 in SEQ ID NO:1; and this amino acid sequence is:

(SEQ ID NO:17)

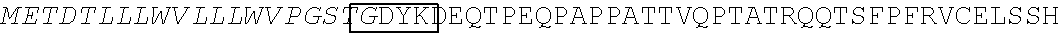

GDLFRFSSDIQCPSFGTRENHTEGLLMVFKDNIIPYSFKVRSYTKIVTNILIYNGHRAD
SVTNRHEEKFSVDSYETDQMDTIYQCYNAVKMTKDGLTRVYVDRDGVNITVNLKPT
GGLANGVRRYASQTELYDAPGRVEATYRTRTTVNCLITDMMAKSNSPFDFFVTTTGQ
TVEMSPFYDGKNKETFHERADSFHVRTNYKIVDYDNRGTNPQGERRAFLDKGTYTL
SWKLENRTAYCPLQHWQTFDSTIATETGKSIHFVTDEGTSCFVTNTTVGIELPDAFKCI
EEQVNKTMHEKYEAVQDRYTKGQEAITYFITSGGLLLAWLPLTPRSLATVKNLTELTT
PTSSPPSSPSPPAPPAARGSTSAAVLRRRRRDVGNATTPVPPAAPGKSLGTLNNPATVQI
QFAYDSLRRQINRMLGDLCRAWCLEQKRQNMVLRELTKINPTTVMSSIYGKAVAAK
RLGDVISVSQCVPVNQATVTLRKSMRVPGSETMCYSRPLVSFSFINDTKTYEGQLGTD
NEIFLTKKMTEVCQATSQYYFQSGNEIHVYNDYHHFKTIELDGIATLQTFISLNTSLIEN

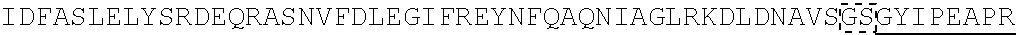

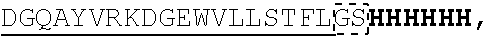

which, relative to the native EctoS sequence, further contains:
at the N-terminus, an Ig Kappa signal peptide sequence in positions 1-20 herein (in non-bolded italics) and a short polypeptide marker sequence in positions 21-25 herein (boxed in solid line), and
at the C-terminus, a fibritin foldon trimerization domain in positions 689-715 herein (underlined), a glycine-serine (GS) flexible linker (boxed in dash line) flanking the fibritin foldon trimerization domain, and a poly histidine affinity tag in positions 718-723 herein (in bold, non-italicized and non-enlarged font size).
18. Amino acid sequence of a polypeptide coined “Ecto_EBV_gB_ectoS_G322C_D478C-Foldon”, comprising an altered EctoS sequence that has the fusion loop substitutions (shown in bolded italics) and two engineered sulfhydryl-containing amino acid residues (shown in bold and an increased font size), relative to the native EctoS sequence, wherein the two engineered sulfhydryl-containing amino acid residues are in positions 325 and 481 herein and are substitutions of cysteine amino acid residues for wild type residues G322 and D478 in SEQ ID NO:1; and this amino acid sequence is:

(SEQ ID NO: 18)

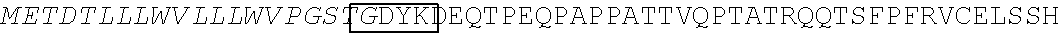

GDLFRFSSDIQCPSFGTRENHTEGLLMVFKDNIIPYSFKVRSYTKIVTNILIYNGHRAD
SVTNRHEEKFSVDSYETDQMDTIYQCYNAVKMTKDGLTRVYVDRDGVNITVNLKPT
GGLANGVRRYASQTELYDAPGRVEATYRTRTTVNCLITDMMAKSNSPFDFFVTTTGQ
TVEMSPFYDGKNKETFHERADSFHVRTNYKIVDYDNRGTNPQGERRAFLDKGTYTL
SWKLENRTAYCPLQHWQTFDSTIATETGKSIHFVTDECTSSFVTNTTVGIELPDAFKCI
EEQVNKTMHEKYEAVQDRYTKGQEAITYFITSGGLLLAWLPLTPRSLATVKNLTELTT
PTSSPPSSPSPPAPPAARGSTSAAVLRRRRRDVGNATTPVPPAAPGKSLGTLNNPATVQI
QFAYDSLRRQINRMLGCLARAWCLEQKRQNMVLRELTKINPTTVMSSIYGKAVAAK
RLGDVISVSQCVPVNQATVTLRKSMRVPGSETMCYSRPLVSFSFINDTKTYEGQLGTD
NEIFLTKKMTEVCQATSQYYFQSGNEIHVYNDYHHFKTIELDGIATLQTFISLNTSLIEN

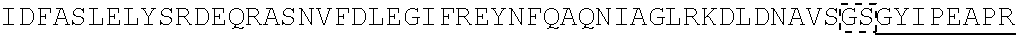

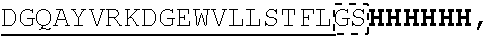

which, relative to the native EctoS sequence, further contains:
at the N-terminus, an Ig Kappa signal peptide sequence in positions 1-20 herein (in non-bolded italics) and a short polypeptide marker sequence in positions 21-25 herein (boxed in solid line), and
at the C-terminus, a fibritin foldon trimerization domain in positions 689-715 herein (underlined), a glycine-serine (GS) flexible linker (boxed in dash line) flanking the fibritin foldon trimerization domain, and a poly histidine affinity tag in positions 718-723 herein (in bold, non-italicized and non-enlarged font size).
19. Amino acid sequence of a polypeptide coined “Ecto_EBV_gB_ectoS_A175C_V178C-Foldon”, comprising an altered EctoS sequence that has the fusion loop substitutions (shown in bolded italics) and two engineered sulfhydryl-containing amino acid residues (shown in bold and an increased font size), relative to the native EctoS sequence, wherein the two engineered sulfhydryl-containing amino acid residues are in positions 178 and 181 herein and are substitutions of cysteine amino acid residues for wild type residues A175 and V178 in SEQ ID NO:1; and this amino acid sequence is:

(SEQ ID NO:19)

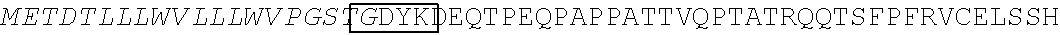

GDLFRFSSDIQCPSFGTRENHTEGLLMVFKDNIIPYSFKVRSYTKIVTNILIYNGHRAD
SVTNRHEEKFSVDSYETDQMDTIYQCYNAVKMTKDGLTRVYVDRDGVNITVNLKPT
GGLCNGCRRYASQTELYDAPGRVEATYRTRTTVNCLITDMMAKSNSPFDFFVTTTG
QTVEMSPFYDGKNKETFHERADSFHVRTNYKIVDYDNRGTNPQGERRAFLDKGTYT
LSWKLENRTAYCPLQHWQTFDSTIATETGKSIHFVTDEGTSSFVTNTTVGIELPDAFKC
IEEQVNKTMHEKYEAVQDRYTKGQEAITYFITSGGLLLAWLPLTPRSLATVKNLTELT
TPTSSPPSSPSPPAPPAARGSTSAAVLRRRRRDVGNATTPVPPAAPGKSLGTLNNPATV
QIQFAYDSLRRQINRMLGDLARAWCLEQKRQNMVLRELTKINPTTVMSSIYGKAVAA
KRLGDVISVSQCVPVNQATVTLRKSMRVPGSETMCYSRPLVSFSFINDTKTYEGQLGT
DNEIFLTKKMTEVCQATSQYYFQSGNEIHVYNDYHHFKTIELDGIATLQTFISLNTSLIE

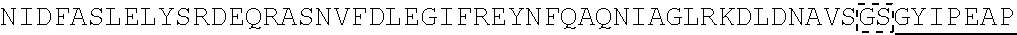

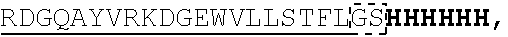

which, relative to the native EctoS sequence, further contains:
at the N-terminus, an Ig Kappa signal peptide sequence in positions 1-20 herein (in non-bolded italics) and a short polypeptide marker sequence in positions 21-25 herein (boxed in solid line), and
at the C-terminus, a fibritin foldon trimerization domain in positions 689-715 herein (underlined), a glycine-serine (GS) flexible linker (boxed in dash line) flanking the fibritin foldon trimerization domain, and a poly histidine affinity tag in positions 718-723 herein (in bold, non-italicized and non-enlarged font size).
20. Amino acid sequence of a polypeptide coined “Ecto_EBV_gB_ectoS_G172C_D564C-Foldon”, comprising an altered EctoS sequence that has the fusion loop substitutions (shown in bolded italics) and two engineered sulfhydryl-containing amino acid residues (shown in bold and an increased font size), relative to the native EctoS sequence, wherein the two engineered sulfhydryl-containing amino acid residues are in positions 175 and 567 herein and are substitutions of cysteine amino acid residues for wild type residues G172 and D564 in SEQ ID NO:1; and this amino acid sequence is:

(SEQ ID NO:20)

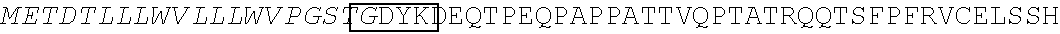

GDLFRFSSDIQCPSFGTRENHTEGLLMVFKDNIIPYSFKVRSYTKIVTNILIYNGHRAD
SVTNRHEEKFSVDSYETDQMDTIYQCYNAVKMTKDGLTRVYVDRDGVNITVNLKPT
CGLANGVRRYASQTELYDAPGRVEATYRTRTTVNCLITDMMAKSNSPFDFFVTTTGQ
TVEMSPFYDGKNKETFHERADSFHVRTNYKIVDYDNRGTNPQGERRAFLDKGTYTL
SWKLENRTAYCPLQHWQTFDSTIATETGKSIHFVTDEGTSSFVTNTTVGIELPDAFKCI
EEQVNKTMHEKYEAVQDRYTKGQEAITYFITSGGLLLAWLPLTPRSLATVKNLTELTT
PTSSPPSSPSPPAPPAARGSTSAAVLRRRRRDVGNATTPVPPAAPGKSLGTLNNPATVQI
QFAYDSLRRQINRMLGDLARAWCLEQKRQNMVLRELTKINPTTVMSSIYGKAVAAK
RLGDVISVSQCVPVNQATVTLRKSMRVPGSETMCYSRPLVSFSFINCTKTYEGQLGT
DNEIFLTKKMTEVCQATSQYYFQSGNEIHVYNDYHHFKTIELDGIATLQTFISLNTSLIE

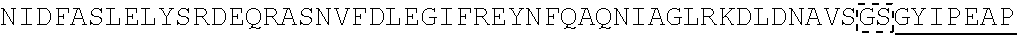

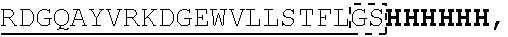

which, relative to the native EctoS sequence, further contains:
at the N-terminus, an Ig Kappa signal peptide sequence in positions 1-20 herein (in non-bolded italics) and a short polypeptide marker sequence in positions 21-25 herein (boxed in solid line), and
at the C-terminus, a fibritin foldon trimerization domain in positions 689-715 herein (underlined), a glycine-serine (GS) flexible linker (boxed in dash line) flanking the fibritin foldon trimerization domain, and a poly histidine affinity tag in positions 718-723 herein (in bold, non-italicized and non-enlarged font size).
21. Amino acid sequence of a polypeptide coined “Ecto_EBV_gB_ectoS_L580C_Y644C-Foldon”, comprising an altered EctoS sequence that has the fusion loop substitutions (shown in bolded italics) and two engineered sulfhydryl-containing amino acid residues (shown in bold and an increased font size), relative to the native EctoS sequence, wherein the two engineered sulfhydryl-containing amino acid residues are in positions 583 and 647 herein and are substitutions of cysteine amino acid residues for wild type residues L580 and Y644 in SEQ ID NO:1; and this amino acid sequence is:

(SEQ ID NO:21)

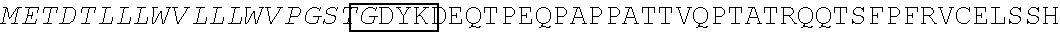

GDLFRFSSDIQCPSFGTRENHTEGLLMVFKDNIIPYSFKVRSYTKIVTNILIYNGHRAD
SVTNRHEEKFSVDSYETDQMDTIYQCYNAVKMTKDGLTRVYVDRDGVNITVNLKPT
GGLANGVRRYASQTELYDAPGRVEATYRTRTTVNCLITDMMAKSNSPFDFFVTTTGQ
TVEMSPFYDGKNKETFHERADSFHVRTNYKIVDYDNRGTNPQGERRAFLDKGTYTL
SWKLENRTAYCPLQHWQTFDSTIATETGKSIHFVTDEGTSSFVTNTTVGIELPDAFKCI
EEQVNKTMHEKYEAVQDRYTKGQEAITYFITSGGLLLAWLPLTPRSLATVKNLTELTT
PTSSPPSSPSPPAPPAARGSTSAAVLRRRRRDVGNATTPVPPAAPGKSLGTLNNPATVQI
QFAYDSLRRQINRMLGDLARAWCLEQKRQNMVLRELTKINPTTVMSSIYGKAVAAK
RLGDVISVSQCVPVNQATVTLRKSMRVPGSETMCYSRPLVSFSFINDTKTYEGQLGTD
NEIFCTKKMTEVCQATSQYYFQSGNEIHVYNDYHHFKTIELDGIATLQTFISLNTSLIE

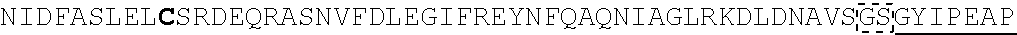

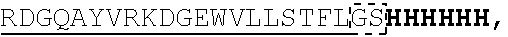

which, relative to the native EctoS sequence, further contains:
at the N-terminus, an Ig Kappa signal peptide sequence in positions 1-20 herein (in non-bolded italics) and a short polypeptide marker sequence in positions 21-25 herein (boxed in solid line), and
at the C-terminus, a fibritin foldon trimerization domain in positions 689-715 herein (underlined), a glycine-serine (GS) flexible linker (boxed in dash line) flanking the fibritin foldon trimerization domain, and a poly histidine affinity tag in positions 718-723 herein (in bold, non-italicized and non-enlarged font size).
22. Amino acid sequence of a polypeptide coined “Ecto_EBV_gB_ectoS_Foldon-DS variants-FLS”, comprising an altered EctoS sequence that has the fusion loop substitutions (“FLS”) (shown in bolded italics); and this amino acid sequence is:

(SEQ ID NO:13)

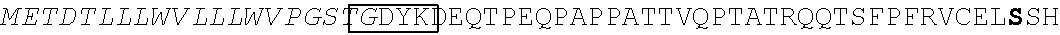

GDLFRFSSDIQCPSFGTRENHTEGLLMVFKDNIIPYSFKVRSYTKIVTNILIYNGHRAD
SVTNRHEEKFSVDSYETDQMDTIYQCYNAVKMTKDGLTRVYVDRDGVNITVNLKPT
GGLANGVRRYASQTELYDAPGRVEATYRTRTTVNCLITDMMAKSNSPFDFFVTTTG
QTVEMSPFYDGKNKETFHERADSFHVRTNYKIVDYDNRGTNPQGERRAFLDKGTYT
LSWKLENRTAYCPLQHWQTFDSTIATETGKSIHFVTDEGTSSFVTNTTVGIELPDAFK
CIEEQVNKTMHEKYEAVQDRYTKGQEAITYFITSGGLLLAWLPLTPRSLATVKNLTEL
TTPTSSPPSSPSPPAPPAARGSTSAAVLRRRRRDVGNATTPVPPAAPGKSLGTLNNPATV
QIQFAYDSLRRQINRMLGDLARAWCLEQKRQNMVLRELTKINPTTVMSSIYGKAVA
AKRLGDVISVSQCVPVNQATVTLRKSMRVPGSETMCYSRPLVSFSFINDTKTYEGQL
GTDNEIFLTKKMTEVCQATSQYYFQSGNEIHVYNDYHHFKTIELDGIATLQTFISLNT

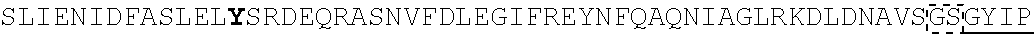

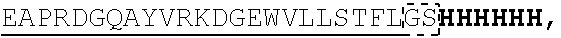

which, relative to the native EctoS sequence, further contains:
at the N-terminus, an Ig Kappa signal peptide sequence in positions 1-20 herein (in non-bolded italics) and a short polypeptide marker sequence in positions 21-25 herein (boxed in solid line), and
at the C-terminus, a fibritin foldon trimerization domain in positions 689-715 herein (underlined), a glycine-serine (GS) flexible linker (boxed in dashed line) flanking the fibritin foldon trimerization domain, and a poly histidine affinity tag in positions 718-723 herein (in bold, non-italicized and non-enlarged font size); and wherein amino acid residues herein suitable for mutations to, or replacement with, cysteine (preferably in pairs for disulfide bond formation, “DS variants”) to stabilize prefusion conformation are shown in bold and an increased font size.
23. Amino acid sequence of a polypeptide coined “Ecto_EBV_gB_ectoS_Foldon-DS variants”, comprising a native EctoS sequence (including the wildtype fusion loop residues WY¹¹⁵⁻¹¹⁶ and WLIW¹⁹⁶⁻¹⁹⁹ (SEQ ID NO:28) herein (shown in bolded italics in this sequence), which correspond to positions 112-113 and 193-196 in SEQ ID NO:1); and this amino acid sequence is:

(SEQ ID NO:22)

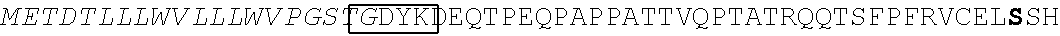

GDLFRFSSDIQCPSFGTRENHTEGLLMVFKDNIIPYSFKVRSYTKIVTNILIYNGWYAD
SVTNRHEEKFSVDSYETDQMDTIYQCYNAVKMTKDGLTRVYVDRDGVNITVNLKPT
GGLANGVRRYASQTELYDAPGWLIWTYRTRTTVNCLITDMMAKSNSPFDFFVTTTG
QTVEMSPFYDGKNKETFHERADSFHVRTNYKIVDYDNRGTNPQGERRAFLDKGTYT
LSWKLENRTAYCPLQHWQTFDSTIATETGKSIHFVTDEGTSSFVTNTTVGIELPDAFK
CIEEQVNKTMHEKYEAVQDRYTKGQEAITYFITSGGLLLAWLPLTPRSLATVKNLTEL
TTPTSSPPSSPSPPAPPAARGSTSAAVLRRRRRDVGNATTPVPPAAPGKSLGTLNNPATV
QIQFAYDSLRRQINRMLGDLARAWCLEQKRQNMVLRELTKINPTTVMSSIYGKAVA
AKRLGDVISVSQCVPVNQATVTLRKSMRVPGSETMCYSRPLVSFSFINDTKTYEGQL
GTDNEIFLTKKMTEVCQATSQYYFQSGNEIHVYNDYHHFKTIELDGIATLQTFISLNT

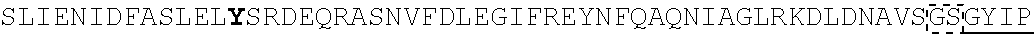

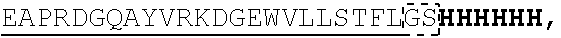

which, relative to the native EctoS sequence, further contains:
at the N-terminus, an Ig Kappa signal peptide sequence in positions 1-20 herein (in non-bolded italics) and a short polypeptide marker sequence in positions 21-25 herein (boxed in solid line), and
at the C-terminus, a fibritin foldon trimerization domain in positions 689-715 herein (underlined), a glycine-serine (GS) flexible linker (boxed in dash line) flanking the fibritin foldon trimerization domain, and a poly histidine affinity tag in positions 718-723 herein (in bold, non-italicized and non-enlarged font size), and wherein amino acid residues herein suitable for mutations to, or replacement with, cysteine (preferably in pairs for disulfide bond formation, “DS variants”) to stabilize prefusion conformation are shown in bold and an increased font size.
24. Amino acid sequence of a polypeptide coined “Ecto_EBV_gB_ectoS_FLM_A175C_V529C_G322C_A480C-Foldon”, comprising an altered EctoS sequence that has the fusion loop substitutions (shown in bolded italics, also called fusion loop mutation, “FLM”) and four engineered sulfhydryl-containing amino acid residues (shown in bold and an increased font size)-2 pairs of disulfide bond bridges, relative to the native EctoS sequence, wherein the four engineered sulfhydryl-containing amino acid residues are in positions 178 and 532, and 325 and 483, herein and are substitutions of cysteine amino acid residues for wild type residues A175 and V529, and wild type residues G322 and A480, in SEQ ID NO:1; and this amino acid sequence is:

(SEQ ID NO: 23)
METDTLLLWVLLLWVPGSTGDYKDEQTPEQPAPPATTVQPTATRQ
QTSFPFRVCELSSHGDLFRFSSDIQCPSFGTRENHTEGLLMVFKD
NIIPYSFKVRSYTKIVTNILIYNGHRADSVTNRHEEKFSVDSYET
DQMDTIYQCYNAVKMTKDGLTRVYVDRDGVNITVNLKPTGGL [symbol] NG
VRRYASQTELYDAPGRVEATYRTRTTVNCLITDMMAKSNSPFDFF
VTTTGQTVEMSPFYDGKNKETFHERADSFHVRTNYKIVDYDNRGT
NPQGERRAFLDKGTYTLSWKLENRTAYCPLQHWQTFDSTIATETG
KSIHFVTDE [symbol] TSSFVTNTTVGIELPDAFKCIEEQVNKTMHEKYEA
VQDRYTKGQEAITYFITSGGLLLAWLPLTPRSLATVKNLTELTTP
TSSPPSSPSPPAPPAARGSTSAAVLRRRRRDVGNATTPVPPAAPG
KSLGTLNNPATVQIQFAYDSLRRQINRMLGDL [symbol] RAWCLEQKRQNM
VLRELTKINPTTVMSSIYGKAVAAKRLGDVISVSQC [symbol] PVNQATVT
LRKSMRVPGSETMCYSRPLVSFSFINDTKTYEGQLGTDNEIFLTK
KMTEVCQATSQYYFQSGNEIHVYNDYHHFKTIELDGIATLQTFIS
LNTSLIENIDFASLELYSRDEQRASNVFDLEGIFREYNFQAQNIA
GLRKDLDNAVSGSGYIPEAPRDGQAYVRKDGEWVLLSTFLGSHHH
HHH,

which, relative to the native EctoS sequence, further contains:
at the N-terminus, an Ig Kappa signal peptide sequence in positions 1-20 herein and a short polypeptide marker sequence in positions 21-25 herein, and
at the C-terminus, a fibritin foldon trimerization domain in positions 689-715 herein, a glycine-serine (GS) flexible linker flanking the fibritin foldon trimerization domain, and a poly histidine affinity tag in positions 718-723 herein.
25. Amino acid sequence of a polypeptide coined “Ecto_EBV_gB_ectoS_FLM_A175C_V529C_G172C_D564C-Foldon”, comprising an altered EctoS sequence that has the fusion loop substitutions (shown in bolded italics, also called fusion loop mutation, “FLM”) and four engineered sulfhydryl-containing amino acid residues (shown in bold and an increased font size)-2 pairs of disulfide bond bridges, relative to the native EctoS sequence, wherein the four engineered sulfhydryl-containing amino acid residues are in positions 178 and 532, and 175 and 567, herein and are substitutions of cysteine amino acid residues for wild type residues A175 and V529, and wild type residues G172 and D564, in SEQ ID NO:1; and this amino acid sequence is:

(SEQ ID NO: 24)
METDTLLLWVLLLWVPGSTGDYKDEQTPEQPAPPATTVQPTATRQ
QTSFPFRVCELSSHGDLFRFSSDIQCPSFGTRENHTEGLLMVFKD
NIIPYSFKVRSYTKIVTNILIYNGHRADSVTNRHEEKFSVDSYET
DQMDTIYQCYNAVKMTKDGLTRVYVDRDGVNITVNLKPT [symbol] GL [symbol] NG
VRRYASQTELYDAPGRVEATYRTRTTVNCLITDMMAKSNSPFDFF
VTTTGQTVEMSPFYDGKNKETFHERADSFHVRTNYKIVDYDNRGT
NPQGERRAFLDKGTYTLSWKLENRTAYCPLQHWQTFDSTIATETG
KSIHFVTDEGTSSFVTNTTVGIELPDAFKCIEEQVNKTMHEKYEA
VQDRYTKGQEAITYFITSGGLLLAWLPLTPRSLATVKNLTELTTP
TSSPPSSPSPPAPPAARGSTSAAVLRRRRRDVGNATTPVPPAAPG
KSLGTLNNPATVQIQFAYDSLRRQINRMLGDLARAWCLEQKRQNM
VLRELTKINPTTVMSSIYGKAVAAKRLGDVISVSQC [symbol] PVNQATVT
LRKSMRVPGSETMCYSRPLVSFSFIN [symbol] TKTYEGQLGTDNEIFLTK
KMTEVCQATSQYYFQSGNEIHVYNDYHHFKTIELDGIATLQTFIS
LNTSLIENIDFASLELYSRDEQRASNVFDLEGIFREYNFQAQNIA
GLRKDLDNAVSGSGYIPEAPRDGQAYVRKDGEWVLLSTFLGSHHH
HHH,

which, relative to the native EctoS sequence, further contains:
at the N-terminus, an Ig Kappa signal peptide sequence in positions 1-20 herein and a short polypeptide marker sequence in positions 21-25 herein, and
at the C-terminus, a fibritin foldon trimerization domain in positions 689-715 herein, a glycine-serine (GS) flexible linker flanking the fibritin foldon trimerization domain, and a poly histidine affinity tag in positions 718-723 herein.
26. Amino acid sequence of a polypeptide coined “Ecto_EBV_gB_ectoS_FLM_G322C_A480C_G172C_D564C-Foldon”, comprising an altered EctoS sequence that has the fusion loop substitutions (shown in bolded italics, also called fusion loop mutation, “FLM”) and four engineered sulfhydryl-containing amino acid residues (shown in bold and an increased font size)-2 pairs of disulfide bond bridges, relative to the native EctoS sequence, wherein the four engineered sulfhydryl-containing amino acid residues are in positions 325 and 483, and 175 and 567, herein and are substitutions of cysteine amino acid residues for wild type residues G³²² and A⁴⁸⁰, and wild type residues G¹⁷² and D⁵⁶⁴, in SEQ ID NO:1; and this amino acid sequence is:

(SEQ ID NO: 25)
METDTLLLWVLLLWVPGSTGDYKDEQTPEQPAPPATTVQPTATRQ
QTSFPFRVCELSSHGDLFRFSSDIQCPSFGTRENHTEGLLMVFKD
NIIPYSFKVRSYTKIVTNILIYNGHRADSVTNRHEEKFSVDSYET
DQMDTIYQCYNAVKMTKDGLTRVYVDRDGVNITVNLKPT [symbol] GLANG
VRRYASQTELYDAPGRVEATYRTRTTVNCLITDMMAKSNSPFDFF
VTTTGQTVEMSPFYDGKNKETFHERADSFHVRTNYKIVDYDNRGT
NPQGERRAFLDKGTYTLSWKLENRTAYCPLQHWQTFDSTIATETG
KSIHFVTDE [symbol] TSSFVTNTTVGIELPDAFKCIEEQVNKTMHEKYEA
VQDRYTKGQEAITYFITSGGLLLAWLPLTPRSLATVKNLTELTTP
TSSPPSSPSPPAPPAARGSTSAAVLRRRRRDVGNATTPVPPAAPG
KSLGTLNNPATVQIQFAYDSLRRQINRMLGDL [symbol] RAWCLEQKRQNM
VLRELTKINPTTVMSSIYGKAVAAKRLGDVISVSQCVPVNQATVT
LRKSMRVPGSETMCYSRPLVSFSFIN [symbol] TKTYEGQLGTDNEIFLTK
KMTEVCQATSQYYFQSGNEIHVYNDYHHFKTIELDGIATLQTFIS
LNTSLIENIDFASLELYSRDEQRASNVFDLEGIFREYNFQAQNIA
GLRKDLDNAVSGSGYIPEAPRDGQAYVRKDGEWVLLSTFLGSHHH
HHH,

which, relative to the native EctoS sequence, further contains:
at the N-terminus, an Ig Kappa signal peptide sequence in positions 1-20 herein and a short polypeptide marker sequence in positions 21-25 herein, and
at the C-terminus, a fibritin foldon trimerization domain in positions 689-715 herein, a glycine-serine (GS) flexible linker flanking the fibritin foldon trimerization domain, and a poly histidine affinity tag in positions 718-723 herein.
27. Amino acid sequence of a polypeptide coined “Ecto_EBV_gB_ectoS_FLM_A175C_V529C_G322C_A480C_G172C_D564C-Foldon”, comprising an altered EctoS sequence that has the fusion loop substitutions (shown in bolded italics, also called fusion loop mutation, “FLM”) and six engineered sulfhydryl-containing amino acid residues (shown in bold and an increased font size)-3 pairs of disulfide bond bridges, relative to the native EctoS sequence, wherein the six engineered sulfhydryl-containing amino acid residues are in positions 178 and 532, 325 and 483, and 175 and 567 herein and are substitutions of cysteine amino acid residues for wild type residues A¹⁷⁵ and V⁵²⁹, wild type residues G³²² and A⁴⁸⁰, and wild type residues G¹⁷² and D⁵⁶⁴ in SEQ ID NO:1; and this amino acid sequence is:

(SEQ ID NO: 26)
METDTLLLWVLLLWVPGSTGDYKDEQTPEQPAPPATTVQPTATRQ
QTSFPFRVCELSSHGDLFRFSSDIQCPSFGTRENHTEGLLMVFKD
NIIPYSFKVRSYTKIVTNILIYNGHRADSVTNRHEEKFSVDSYET
DQMDTIYQCYNAVKMTKDGLTRVYVDRDGVNITVNLKPT [symbol] GL [symbol] NG
VRRYASQTELYDAPGRVEATYRTRTTVNCLITDMMAKSNSPFDFF
VTTTGQTVEMSPFYDGKNKETFHERADSFHVRTNYKIVDYDNRGT
NPQGERRAFLDKGTYTLSWKLENRTAYCPLQHWQTFDSTIATETG
KSIHFVTDE [symbol] TSSFVTNTTVGIELPDAFKCIEEQVNKTMHEKYEA
VQDRYTKGQEAITYFITSGGLLLAWLPLTPRSLATVKNLTELTTP
TSSPPSSPSPPAPPAARGSTSAAVLRRRRRDVGNATTPVPPAAPG
KSLGTLNNPATVQIQFAYDSLRRQINRMLGDL [symbol] RAWCLEQKRQNM
VLRELTKINPTTVMSSIYGKAVAAKRLGDVISVSQC [symbol] PVNQATVT
LRKSMRVPGSETMCYSRPLVSFSFIN [symbol] TKTYEGQLGTDNEIFLTK
KMTEVCQATSQYYFQSGNEIHVYNDYHHFKTIELDGIATLQTFIS
LNTSLIENIDFASLELYSRDEQRASNVFDLEGIFREYNFQAQNIA
GLRKDLDNAVSGSGYIPEAPRDGQAYVRKDGEWVLLSTFLGSHHH
HHH,

which, relative to the native EctoS sequence, further contains:
at the N-terminus, an Ig Kappa signal peptide sequence in positions 1-20 herein and a short polypeptide marker sequence in positions 21-25 herein, and
at the C-terminus, a fibritin foldon trimerization domain in positions 689-715 herein, a glycine-serine (GS) flexible linker flanking the fibritin foldon trimerization domain, and a poly histidine affinity tag in positions 718-723 herein.
28 Amino acid sequence of the native EctoS sequence disclosed herein, corresponding to positions 23-683 of SEQ ID NO:1:

(SEQ ID NO: 27)
QTPEQPAPPATTVQPTATRQQTSFPFRVCELSSHGDLFRFSSDIQ
CPSFGTRENHTEGLLMVFKDNIIPYSFKVRSYTKIVTNILIYNGW
YADSVTNRHEEKFSVDSYETDQMDTIYQCYNAVKMTKDGLTRVYV
DRDGVNITVNLKPTGGLANGVRRYASQTELYDAPGWLIWTYRTRT
TVNCLITDMMAKSNSPFDFFVTTTGQTVEMSPFYDGKNKETFHER
ADSFHVRTNYKIVDYDNRGTNPQGERRAFLDKGTYTLSWKLENRT
AYCPLQHWQTFDSTIATETGKSIHFVTDEGTSSFVTNTTVGIELP
DAFKCIEEQVNKTMHEKYEAVQDRYTKGQEAITYFITSGGLLLAW
LPLTPRSLATVKNLTELTTPTSSPPSSPSPPAPSAARGSTPAAVL
RRRRRDAGNATTPVPPTAPGKSLGTLNNPATVQIQFAYDSLRRQI
NRMLGDLARAWCLEQKRQNMVLRELTKINPTTVMSSIYGKAVAAK
RLGDVISVSQCVPVNQATVTLRKSMRVPGSETMCYSRPLVSFSFI
NDTKTYEGQLGTDNEIFLTKKMTEVCQATSQYYFQSGNEIHVYND
YHHFKTIELDGIATLQTFISLNTSLIENIDFASLELYSRDEQRAS
NVFDLEGIFREYNFQAQNIAGLRKDLDNAVS.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A polypeptide derived from the glycoprotein B (gB) of an Epstein-Barr virus (EBV), comprising an altered gB ectodomain sequence that has modifications relative to native gB ectodomain sequence of the EBV, said native gB ectodomain comprising the amino acid sequence set forth as residues 23-683 of SEQ ID NO:1, wherein the modifications comprise substituting one or more pairs of amino acid residues with one or more pairs of engineered sulfhydryl-containing amino acid residues.

2. The polypeptide of claim 1, wherein the substitutions with the one or more pairs of engineered sulfhydryl-containing amino acid residues comprises amino acid substitutions of at least two of S54C, S55C, H56C, G172C, A175C, V178C, G227C, G322C, S325C, D478C, A480C, A515C, V529C, D564C, L580C, Y644C, L696C, and S727C compared to SEQ ID NO:1, wherein the amino acid numbering is based on amino acid positions in SEQ ID NO:1.

3. The polypeptide of claim 2, wherein the modifications further comprise amino acid substitutions at one or more positions selected from W112, Y113, W193, L194, 1195, and W196 based on amino acid positions in SEQ ID NO:1, and optionally wherein wild type residues WY¹¹²⁻¹¹³ and WLIW¹⁹³⁻¹⁹⁶ (SEQ ID NO:28) in SEQ ID NO: 1 are replaced with HR and RVEA (SEQ ID NO:29).

4. The polypeptide of claim 1, further comprising all or one or more of:
a signal sequence on the N-terminus end relative to the altered gB ectodomain sequence,
a trimerization domain on the C-terminus end relative to the altered gB ectodomain sequence,
a poly histidine affinity tag, and
one or more linker sequences;
and optionally wherein the polypeptide is in a configuration from the N-terminal to C-terminal direction: signal sequence-altered gB ectodomain sequence-linker sequence 1-trimerization domain-linker sequence 2-poly histidine affinity tag.

5. The polypeptide of claim 4, wherein the signal sequence comprises an amino acid sequence of METDTLLLWVLLLWVPGSTG (SEQ ID NO:31), and wherein the trimerization domain comprises a T4 fibritin trimerization domain.

6. The polypeptide of claim 1, wherein the substitutions with the one or more pairs of engineered sulfhydryl-containing amino acid residues comprise amino acid substitutions of S54C and A515C, S55C and G227C, H56C and G227C, A175C and V529C, G322C and A480C, S325C and A480C, G322C and D478C, L696C and S727C, A175C and V178C, G172C and D564C, or L580C and Y644C, compared to SEQ ID NO:1 based on amino acid positions in SEQ ID NO:1.

7. (canceled)

8. (canceled)

9. (canceled)

10. (canceled)

11. (canceled)

12. (canceled)

13. (canceled)

14. (canceled)

15. (canceled)

16. (canceled)

17. The polypeptide of claim 1, wherein the substitutions with the one or more pairs of engineered sulfhydryl-containing amino acid residues comprise one or more pairs of amino acid substitutions, compared to SEQ ID NO: 1 based on amino acid positions in SEQ ID NO:1, selected from:
S54C and A515C,
S55C and G227C,
H56C and G227C,
A175C and V529C,
G322C and A480C,
S325C and A480C,
G322C and D478C,
L696C and S727C,
A175C and V178C,
G172C and D564C, and
L580C and Y644C.

18. The polypeptide of claim 17, wherein the substitutions comprise two or more pairs of the amino acid substitutions selected from A175C and V529C, G322C and A480C, and G172C and D564C.

19. The polypeptide of claim 1, wherein at least one native cysteine amino acid residue in the native gB ectodomain sequence is substituted with another amino acid residue selected from the group of Ala, Arg, As, Asp, Glu, Gln, Gly, His, Ile, Leu, Lys, Met, Phe, Pro, Ser, Thr, Trp, Tyr, and Val.

20. The polypeptide of claim 1, wherein the modifications further comprise substituting one or more proline amino acid residues at a position selected from one or more of R515, H516, and V517 compared to SEQ ID NO:1 based on amino acid positions in SEQ ID NO:1.

21. The polypeptide of claim 1, comprising the altered gB ectodomain sequence that is otherwise identical or a conservatively modified variant to the native gB ectodomain sequence except for the modifications, wherein the modifications consist of:
(i) one or more pairs of amino acid substitutions, compared to SEQ ID NO: 1 based on amino acid positions in SEQ ID NO:1, selected from S54C and A515C, S55C and G227C, H56C and G227C, A175C and V529C, G322C and A480C, S325C and A480C, G322C and D478C, L696C and S727C, A175C and V178C, G172C and D564C, and L580C and Y644C; or
(ii) the one or more pairs of the amino acid substitutions of (i) and amino acid substitutions at one or more positions selected from W112, Y113, W193, L194, 1195, and W196 based on amino acid positions in SEQ ID NO:1.

22. A protein comprising three copies of a polypeptide of claim 1.

23. The protein of claim 22, wherein each copy of the polypeptide comprises a trimerization domain on the C-terminus end relative to the altered gB ectodomain sequence.

24. The protein of claim 22, further comprising an additional glycoprotein, wherein the additional glycoprotein comprises EBV gHgL, gp42, gp350, gp220, complex gp350/220, or a combination thereof.

25. (canceled)

26. A nucleic acid molecule encoding the polypeptide of claim 1.

27. The nucleic acid molecule of claim 26, which is a messenger ribonucleic acid (mRNA) comprising an open reading frame encoding the polypeptide.

28. A vector comprising the nucleic acid molecule of claim 26 or 27.

29. An isolated host cell comprising the vector of claim 28.

30. An immunogenic composition, comprising a first component comprising the polypeptide of claim 1, a nucleic acid molecule encoding the polypeptide, or both, and a second component comprising a pharmaceutically acceptable carrier.

31. The immunogenic composition of claim 30, further comprising a third component, wherein the third component comprises a nucleic acid molecule encoding another polypeptide of claim 1 with a sequence that does not match the polypeptide used or encoded by in the first component.

32. The immunogenic composition of claim 30, wherein the first component comprises the nucleic acid molecule encoding the polypeptide, and the nucleic acid molecule is an mRNA comprising an open reading frame encoding the polypeptide.

33. An immunogenic composition, comprising a first component comprising the protein of claim 22, and a second component comprising a pharmaceutically acceptable carrier.

34. A method to induce an immunogenic response to a target Epstein-Barr virus protein, the method comprising administering the polypeptide of claim 1 to a subject in need thereof.

35. A method to induce an immunogenic response to a target Epstein-Barr virus protein, the method comprising administering the immunogenic composition of claim 32 to a subject in need thereof.


SEQUENCE LISTING (54 sequences)

SEQ ID NO: 1 - protein, 857 aa, unidentified
  source 1..857: note = Epstein-Barr virus
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 2 - protein, 857 aa, synthetic construct
MTRRRVLSVVVLLAALACRLGAQTPEQPAPPATTVQPTATRQQTSFPFRVCELCSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVCAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 3 - protein, 857 aa, synthetic construct
MTRRRVLSVVVLLAALACRLGAQTPEQPAPPATTVQPTATRQQTSFPFRVCELSCHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTCQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 4 - protein, 857 aa, synthetic construct
MTRRRVLSVVVLLAALACRLGAQTPEQPAPPATTVQPTATRQQTSFPFRVCELSSCGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTCQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 5 - protein, 857 aa, synthetic construct
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLCNGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCCPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 6 - protein, 857 aa, synthetic construct
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDECTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLCRAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 7 - protein, 857 aa, synthetic construct
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSCFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLCRAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 8 - protein, 857 aa, synthetic construct
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDECTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGCLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 9 - protein, 857 aa, synthetic construct
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGECMDSLGSVGQSITNLVSTVGGLFSSLVSGFICFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 10 - protein, 857 aa, synthetic construct
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLCNGCRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 11 - protein, 857 aa, synthetic construct
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTCGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINCTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 12 - protein, 857 aa, synthetic construct
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFCTKKMTEVCQATSQYYFQSGNEIHVYNDYHHFKTIELDGIATLQTFISLNTSLIENIDFASLELCSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 13 - protein, 723 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSGYIPEAPRDGQAYVRKDGEWVLLSTFLGSHHHHHH

SEQ ID NO: 14 - protein, 723 aa, synthetic construct
METDTLLLWVLLLWVPGSTGDYKDEQTPEQPAPPATTVQPTATRQQTSFPFRVCELCSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVCAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSGYIPEAPRDGQAYVRKDGEWVLLSTFLGSHHHHHH

SEQ ID NO: 15 - protein, 723 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLCNGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCCPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSGYIPEAPRDGQAYVRKDGEWVLLSTFLGSHHHHHH

SEQ ID NO: 16 - protein, 723 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDECTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLCRAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSGYIPEAPRDGQAYVRKDGEWVLLSTFLGSHHHHHH

SEQ ID NO: 17 - protein, 723 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSCFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLCRAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSGYIPEAPRDGQAYVRKDGEWVLLSTFLGSHHHHHH

SEQ ID NO: 18 - protein, 723 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDECTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGCLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSGYIPEAPRDGQAYVRKDGEWVLLSTFLGSHHHHHH

SEQ ID NO: 19 - protein, 723 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLCNGCRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSGYIPEAPRDGQAYVRKDGEWVLLSTFLGSHHHHHH

SEQ ID NO: 20 - protein, 723 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTCGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINCTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSGYIPEAPRDGQAYVRKDGEWVLLSTFLGSHHHHHH

SEQ ID NO: 21 - protein, 723 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFCTKKMTEVCQATSQYYFQSGNEIHVYNDYHHFKTIELDGIATLQTFISLNTSLIENIDFASLELCSRDEQRASNVFDLEGIFREYNFQAQNIAGLRKDLDNAVSGSGYIPEAPRDGQAYVRKDGEWVLLSTFLGSHHHHHH

SEQ ID NO: 22 - protein, 723 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSGYIPEAPRDGQAYVRKDGEWVLLSTFLGSHHHHHH

SEQ ID NO: 23 - protein, 723 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLCNGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDECTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLCRAWCLEQKRQNMVLRELTKINPTTVMSSIYGKAVAAKRLGDVISVSQCCPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSGYIPEAPRDGQAYVRKDGEWVLLSTFLGSHHHHHH

SEQ ID NO: 24 - protein, 723 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTCGLCNGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCCPVNQATVTLRKSMRVPGSETMCYSRPLVSFSFINCTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSGYIPEAPRDGQAYVRKDGEWVLLSTFLGSHHHHHH

SEQ ID NO: 25 - protein, 723 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTCGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDECTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLCRAWCLEQKRQNMVLRELTKINPTTVMSSIYGKAVAAKRLGDVISVSQCVPVNQATVTLRKSMRVPGSETMCYSRPLVSFSFINCTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSGYIPEAPRDGQAYVRKDGEWVLLSTFLGSHHHHHH

SEQ ID NO: 26 - protein, 723 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTCGLCNGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDECTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLCRAWCLEQKRQNMVLRELTKINPTTVMSSIYGKAVAAKRLGDVISVSQCCPVNQATVTLRKSMRVPGSETMCYSRPLVSFSFINCTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSGYIPEAPRDGQAYVRKDGEWVLLSTFLGSHHHHHH

SEQ ID NO: 27 - protein, 661 aa, unidentified
  source 1..661: note = Epstein-Barr virus
QTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVS

SEQ ID NO: 28 - protein, 4 aa, synthetic construct
WLIW

SEQ ID NO: 29 - protein, 4 aa, synthetic construct
RVEA

SEQ ID NO: 30 - protein, 5 aa, synthetic construct
DYKDE

SEQ ID NO: 31 - protein, 20 aa, synthetic construct
METDTLLLWVLLLWVPGSTG

SEQ ID NO: 32 - protein, 27 aa, synthetic construct
GYIPEAPRDGQAYVRKDGEWVLLSTFL

SEQ ID NO: 33 - protein, 25 aa, synthetic construct
MGVLLTQRTLLSLVLALLFPSMASM

SEQ ID NO: 34 - protein, 16 aa, synthetic construct
MKCLLYLAFLFIGVNC

SEQ ID NO: 35 - protein, 19 aa, synthetic construct
MGWSCIILFLVATATGVHS

SEQ ID NO: 36 - protein, 17 aa, synthetic construct
MRAWIFFLLCLAGRALA

SEQ ID NO: 37 - protein, 21 aa, synthetic construct
MWWRLWWLLLLLLLLWPMVWA

SEQ ID NO: 38 - protein, 22 aa, synthetic construct
MDMRVPAQLLGLLLLWLRGARC

SEQ ID NO: 39 - protein, 16 aa, synthetic construct
MPLLLLLPLLWAGALA

SEQ ID NO: 40 - protein, 23 aa, synthetic construct
MDAMKRGLCCVLLLCGAVFVSPS

SEQ ID NO: 41 - protein, 18 aa, synthetic construct
MAFLWLLSCWALLGTTFG

SEQ ID NO: 42 - protein, 15 aa, synthetic construct
MNLLLILTFVAAAVA

SEQ ID NO: 43 - protein, 20 aa, synthetic construct
MYRMQLLSCIALSLALVTNS

SEQ ID NO: 44 - protein, 17 aa, synthetic construct
MGVKVLFALICIAVAEA

SEQ ID NO: 45 - protein, 16 aa, synthetic construct
MKWVTFISLLFSSAYS

SEQ ID NO: 46 - protein, 16 aa, synthetic construct
MKTIIALSYIFCLVLG

SEQ ID NO: 47 - protein, 24 aa, synthetic construct
MALWMRLLPLLALLALWGPDPAAA

SEQ ID NO: 48 - protein, 16 aa, synthetic construct
MKPIFLVLLVVTSAYA

SEQ ID NO: 49 - protein, 5 aa, synthetic construct
GGGGS

SEQ ID NO: 50 - protein, 33 aa, synthetic construct
RMKQIEDKIEEILSKIYHIENEIARIKKLIGER

SEQ ID NO: 51 - protein, 55 aa, synthetic construct
GSSGVRLWATRQAMLGQVHEVPEGWLIFVAEQEELYVRVQNGFRKVQLEARTPLP

SEQ ID NO: 52 - protein, 119 aa, synthetic construct
EEAELAYLLGELAYKLGEYRIAIRAYRIALKRDPNNAEAWYNLGNAYYKQGDYDEAIEYYQKALELDPNNAEAWYNLGNAYYKQGDYDEAIEYYQKALELDPNNAEAKQNLGNAKQKQG

SEQ ID NO: 53 - protein, 35 aa, synthetic construct
XRMKQLEDKVEELLSKNYHLENEVARLKKLVGERX

SEQ ID NO: 54 - protein, 181 aa, synthetic construct
MGSSHHHHHHSQDPMLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS